(19) United States
(12) Patent Application Publication
(10) Pub. No.: US 2026/0283553 A1
(43) Pub. Date: Sep. 24, 2026

(54) WEARABLE SCREEN-PRINTED BIOSENSOR WITH PAPER MICROFLUIDICS FOR SWEAT ANALYSIS

(71) Applicant: JABIL INC., St. Petersburg, FL (US)

(72) Inventors: JORDYN BIZZELL, St. Petersburg, FL (US); Sai Avuthu, St. Petersburg, FL (US)

(21) Appl. No.: 19/490,972

(22) PCT Filed: Jun. 13, 2024
(86) PCT No.: PCT/US2024/033712

§ 371 Date: Dec. 8, 2025

Related U.S. Application Data
(60) Provisional application No. 63/521,621, filed on Jun. 16, 2023.

(51) Int. Cl. A61B 5/00; A61B 5/145; A61B 5/1477; A61B 10/00

(52) U.S. Cl. CPC A61B 5/6833; A61B 5/0002; A61B 5/14521; A61B 5/1477; A61B 5/4875; A61B 10/0064; A61B 2560/0214; A61B 2560/0412

(57) ABSTRACT

An apparatus, system and method for providing a hydration detection device. The hydration detection device, includes: a flexible substrate having screen-printed therein an electrical circuit comprising a plurality of layers of conductive and dielectric inks; a sensor upon the flexible substrate, wherein the sensor is capable of detecting element data indicative of characteristics of sweat; untreated chromatographic paper placed so as to contact skin of a user, and capable of wicking the sweat from the skin to the sensor along a micro-sized fluidic channel; and a bluetooth antenna on the flexible substrate and communicative with the sensor, and capable of communicating the element data wirelessly to an application capable of indicating to the user a propriety of the hydration of the user based upon the element data.

### CROSS-REFERENCE TO RELATED APPLICATIONS

This application claims priority to U.S. provisional application No. 63/521,621, entitled: “Wearable Screen-Printed Biosensor with Paper Microfluidics for Sweat Analysis,” filed Jun. 16, 2023, the contents of which are incorporated herein by reference in its entirety.

### FIELD OF THE DISCLOSURE

The disclosure is directed generally to wearable devices and, more particularly, for an apparatus, system and method of providing a wearable screen-printed biosensor with paper microfluidics for sweat analysis.

### BACKGROUND

Dehydration is a condition that is treatable with great success if detected early. Recently, there has been growing interest in analyzing sweat to gain insight into human health, especially hydration levels.

Younger children, who can suffer water and sodium imbalances simply from nausea, and older individuals 55 and older and who may suffer from comorbidities, are the most prone to dehydration. Traditional treatments include the use of oral rehydration solution (ORS), saline or glucose infusion for severe cases. The conditions that occur most often with dehydration are sunstroke and electrolyte imbalance.

The monitoring of electrolyte imbalance is most applicable to the condition of dehydration. Dehydration is a condition caused by loss of water or salt in the body. It has several potential causes including disease, effects from medications, heat exposure, and physical activity. Adults are not likely to become dehydrated long-term unless exposed to an illness. Chronic dehydration is much more common in the senior-aged population.

Dehydration is a condition that has the ability to significantly impact the body's organs, depending on the severity. Up to 60% of the human body is composed of water. The brain and heart are composed 73% by water, and the lungs are about 83% water. The skin also contains roughly 64% water, while muscles and kidneys are composed of 79% water.

The primary uses of water by the body are the regulation of temperature, maintenance of overall homeostasis, and proper functioning of organs. For the kidney, water helps keep blood vessels open for blood transportation, which is important for the effective transfer of nutrients throughout the body. Water is also utilized by the kidneys in the removal of waste from the blood. If an individual is dehydrated, the kidneys are unable to perform this function, which can cause waste to build up and cause the formation of kidney stones.

The heart is also largely impacted by dehydration. Under normal conditions, the blood flow is regular, which helps keep the heart rate normal. However, if water levels drop, the blood circulates slower and the heart attempts to compensate by beating faster.

Water also aids the eccrine and apocrine glands in the removal of waste in the form of sweat instead. Sweat is a mixture of 99% water and 1% chemicals, and thus hydration is key for the body to be able to produce sweat for the secretion of toxins.

Homeostatic mechanisms maintain about 66% of water content intracellularly and 33% extracellularly, of which 20% is intravascular. Euvolemic processes maintain a steady volume of blood in the body, which can easily be disturbed by fluctuations in water, as plasma constitutes 55% of blood content and is itself composed of around 90% water.

Normally, water homeostasis is regulated by osmoreceptors in the brain. States of dehydration perceived by these receptors typically stimulate thirst in the hypothalamus to increase water intake and remedy any abnormalities. If water is not consumed, the hypothalamus can induce antidiuretic hormone (ADH) release by the posterior pituitary gland, which causes the kidneys to reabsorb more water. This process is induced by decreased blood pressure, which triggers renin secretion from the kidney; renin converts to angiotensin I and then angiotensin II, increasing aldosterone release from adrenal glands, in tum increasing sodium and water absorption from the kidney. Using these methods, body volume, sodium and water concentrations are maintained.

Both insufficient and over-aggressive fluid replacement can be problematic. Hypoperfusion in vital organs and heightened renal efforts to retain fluid can lead to altered mental status, renal failure, shock liver, lactic acidosis, hypotension, and death, as organs fail. Conditions relating to fluid and electrolyte imbalances may also arise, which include: uremia, hyponatremia, hypematremia, hypokalemia, hyperkalemia, metabolic acidosis, and metabolic alkalosis.

Electrolyte balance, such as those of sodium and potassium, can easily be disturbed by hyper or hypo-perfusion, leading to seizures and any number of electrical or biochemical abnormalities due to these ions' roles in maintaining intracellular and extracellular environments. Too much fluid replacement may lead to peripheral and pulmonary edemas, and, in extremely mismanaged cases of severe hyponatremia, central pontine myelinolysis (CPM). CPM is a neurological disorder characterized by a demyelination of nerve cells in the pons, leading to a number of psychiatric and behavioral changes as well as a variety of movement disorders such as mutism, parkinsonism, dystonia, and catatonia.

Patients experiencing dehydration have a higher risk of mortality. Dehydration in elderly patients often causes confusion and aggression, especially if they are also diagnosed with dementia. Serious cases of dehydration cause psychosis, delusions, and unconsciousness.

Dehydration may also cause sunken eyes, dry mouth, tachycardia, headache, lethargy, and dizziness. Dehydration is difficult to diagnose due to symptoms overlapping with other conditions, especially in the elderly. Dehydration may be identified biochemically by testing plasma urea to creatine ratio or serum osmolarity. Dehydration can be identified without lab testing by a skin turgor test, tachycardia, dry mouth, urine color, and physical, such as balance, testing.

The most common outcome experienced by patients because of having dehydration is a healthy recovery, as the disease in remedied very simply and safely just by introducing fluids into the body and restoring the water and electrolyte contents of the blood. In terms of negative outcomes, acute kidney injury occurs in a small percentage of patients with dehydration. Additionally, older hospitalized adults may experience a heightened mortality rate.

Patients who received a dehydration diagnosis during their hospital stay were much more likely to die in the hospital, regardless of any external factors. Concomitant diagnoses of dehydration paired with principal diagnoses spanning a variety of diseases were associated with significantly higher rates of mortality within 30 days to a year. Acute kidney injury (AKI) has a mortality rate of 10-12%, with dehydration being a significant risk factor. In one study, dehydration was found to be the cause of 46% of AKI cases.

Additionally, dehydration following stroke events may cause venous thromboembolism and general thrombosis. Delirium is another possible outcome, as dehydration is found in 66% of delirious patients. Heart failure and other critical illnesses, such as renal failure, are also possible outcomes, though these situations typically involve dehydration as only a minor contributor.

Dehydration causes a significant economic burden, not only on the patients but on the entire health industry. While dehydration is one of the most common preventable diagnoses, it is nevertheless the underlying cause of many hospital admittances. The Agency for Healthcare Research and Quality (AHRQ) estimated that dehydration has cost approximately $1.6 billion in hospital charges within the span of a year.

Hospital charges are associated with two primary expenditures, which are clinical and hospital stay costs. The clinical costs are largely dependent on the utilization of resources which, in some cases, can be an enormous cost due to the many clinical conditions that can arise from dehydration. This also influences longer hospital stays, which unfortunately increases the overall hospital stay costs due to the prolonged use of room and board.

Dehydration hospital admissions and readmissions could be largely diminished with proper preventative treatment and outpatient care. According to the AHRQ, nearly 1 in 10 of the estimated 40 million hospitalizations in 2008 could have been avoided. It has also been calculated that avoiding dehydration with certain preventative or after care measures can lead to health-care industry savings of up to or more than $1 billion. Dehydration is an easily avoidable condition, but can lead to more catastrophic diseases and injuries. For example, a National Pressure Ulcer Long-Term Care Study found that “dehydration was associated with a 42% increase in risk of developing pressure ulcers in nursing home residents. The estimated annual cost for treating pressure, or decubitus, ulcers in the United States (US) is $1.3 billion.”

Thus, the best approach to treating and managing dehydration is prevention. The market has presented many solutions for prevention, including hydration-monitoring devices. For example, a sweat patch uses in-patch channels to receive sweat and, using colorimetric sensors, monitor the content of the sweat. Users are typically instructed to engage in strenuous exercise to provide sweat to the channels. However, the patches are often placed at body locations that produce only minimal sweat, and hence the channels often go un-filled.

Candy-like jelly drops can contain 95 percent water and electrolytes to supplement hydration. However, as these jelly drops are often used with elderly patients suffering from mental acuity issues, or with the very young, it is incumbent on caregivers to provide the jelly drops to avoid dehydration, when and as needed.

Prevention can also be as simple as drinking water or electrolyte-rich beverages. However, for the most at-risk portions of the population, this solution is again dependent on care-givers monitoring water and electrolyte intake, and supplying the requisite hydration when and as needed.

Once dehydration occurs, hydration treatment becomes necessary. For minor dehydration, the primary treatment mechanism is oral rehydration therapy (ORT), in which the patient consume fluids such as water or broth. Healthcare professionals prompt patients to take small sips of such fluids to avoid vomiting, and may employ a nasogastric tube to control consumption. As a result, these scenarios often occur in a healthcare setting, such as a physician's office or hospital, and some medical expertise is required to instruct patients or set up nasogastric feeding.

In cases of severe dehydration or hypovolemic shock, typical treatments include a variety of percutaneous treatments via IV rehydration, ranging from simple saline solution to special solutions such as Ringer's lactate IV fluid, for example. Balancing the patient's fluid intake to the fluid expelled due to vomiting or diarrhea is crucial in cases of dehydration, especially in those cases brought about by disease. These scenarios require extensive medical expertise to safely set up an IV and most often occur in hospital settings, where patients often require attentive monitoring during the rehydration process.

While diagnostic solutions do not directly treat dehydration, this solution type is fundamental in identifying and guiding the development of the most optimal treatment plan by providing an evaluation of the severity of dehydration. Diagnostic solutions for individuals suffering from dehydration come in several different forms. The most common method of diagnosis is through an evaluation by a doctor who performs physical examinations, typically consisting of checking blood pressure and heart rate. Irregular levels and rates, however, can be correlated with a multitude of unrelated diseases, so additional aspects of the individual's physical condition are assessed, including their state of consciousness, degree of sunken eyes, and production of tears and urine. In severe cases, laboratory testing is necessary to identify electrolyte imbalance and determine kidney function.

These metrics are typically determined by administering a basic metabolic panel (BMP), urinalysis or a blood test for analysis of electrolyte concentrations such as sodium and potassium. The BMP test costs around $235. Urinalysis tests generally range from $30 to $250. Analysis of electrolytes from blood tests cost up to $40. These prices are largely determined by the depth of analysis and location at which the samples are analyzed. For example, if tests are completed at an urgent care, doctor's office, or laboratory, the price is generally less expensive than the hospital.

Dehydration can also be detected by testing skin turgor, which involves pinching the skin of the individual and seeing whether it returns to its normal state. The skin's inability to bounce back indicates that the individual is dehydrated because lack of hydration causes the skin to lose elasticity.

An uncommon method of diagnosis uses a wearable sensor to monitor and detect dehydration. While such device monitoring is more beneficial for the prevention of dehydration, this solution can also be used for diagnosis.

The behavioral and lifestyle changes to prevent the onset of dehydration must be implemented by the individual outside of a clinical setting. Drinking adequate amounts of water is the most important preventative measure for an individual to take to prevent dehydration. The Mayo Clinic recommends individuals to consume 3.7 liters of water for men and 2.7 liters of water for women. Consuming extra fluids to replace fluids lost by exercise, vomiting, and diarrhea is another important component of maintaining hydration in daily life.

Young children who are not able to monitor their own hydration should be encouraged to drink water. In cases of illness, children should be admitted to the hospital for clinical treatment. The elderly should be encouraged by caretakers or nursing home staff to consume adequate fluid, because they experience conditions that occur alongside and contribute to dehydration.

The main gap between the desired outcomes and the outcomes achieved by existing solutions lies in preventative measures. Monitoring hydration levels in a portable, non-invasive manner on a large scale that can prevent dehydration from becoming severe in the young, elderly, or athletes is the primary targeted solution. Athletes could also use hydration monitors when working out, especially endurance athletes who exercise for long periods of time. Monitoring hydration in such a way to prevent dehydration from becoming severe and requiring IV fluids and hospital intervention would help create more positive health outcomes and would lower health-care costs.

However, most monitoring devices available are geared towards specific targets, such as athletes, youth, or the elderly, primarily due to these populations' higher susceptibility to developing dehydration. Further, the main issue that arises with dehydration in relation to the young and elderly is that they have a reduced ability to use monitoring devices and/or to rehydrate on their own.

Out of all the stakeholders, patients are the group that prioritize clinical outcomes the most, because it is their health and body that is affected by the condition. Therefore, patients have a personal interest in eliminating symptoms from dehydration, because serious complications for severe dehydration include organ failure, coma, and death. Patients may fear these outcomes and seek preventative measures to prevent serious complications.

However, the costs borne by patients if serious complications do develop is significant. For example, in the event that dehydration advances enough to kidney failure, the estimated cost of treatment is $23,700 per person. For some individuals, severe dehydration leads to cerebral edema, which can be a huge financial strain for patients due to the resources necessitated by the hospital to perform surgical treatment.

Doctors and other medical professionals involved in patient care have a vested interest in clinical outcomes, because their fundamental goal is to cure the patient. Therefore, they are going to select the method of treatment that is the most effective for the patient's needs. Of course, this may lead to the choice of more expensive treatments that may significantly impact the patient's financial well-being after treatment, even if the physical malady has been cured. For example, in 1996, it was reported that $1.36 billion was spent to treat elderly patients that were hospitalized with dehydration and other illnesses that were caused by it.

An insurance company is generally the main financial stakeholder in the healthcare provision process in the United States, but the insurance company holds the least personal interest in clinical outcomes for the patient. For patients with Medicare or Medicaid, this insurance entity is the US government. The primary interest for insurance companies in dehydration treatment is lowering the cost of care. Additionally, insurance companies consider the safety of the treatment in terms of whether there is a risk of leading to more expensive complications. The insurance company thus will typically prefer treatments that coincidentally align with patient, caregiver, or doctor convenience simply because they are easily and cheaply implemented, rendering them a mutually desirable solution for both parties.

Insurance companies suffer major economic burdens due to dehydration, especially in severe cases which develop into more threatening conditions. With these cases, most patients require hospital care, and the average hospitals stays equate to around $11,700 with Medicare, while other insurances can cause the bills to reach up to $12,600.

Next-level treatments vary depending on the severity of the disease beyond a minor case of dehydration, and how long a person waits to seek treatment. In some cases, severe and untreated dehydration can lead to more complicated issues, such as kidney failure. When the condition progresses to this extent, the individual must seek direct treatment from a nephrologist. Individuals suffering from kidney failure can survive a few years at most without dialysis, however the exact time frame varies depending on the individual's stage of kidney failure. In most cases, kidney failure is not curable, but the symptoms can be relieved with proper treatment. Undergoing dialysis would resolve some of the symptoms but can only be done so many times until a transplant is necessary.

Another condition that can arise due to untreated dehydration is hypovolemic shock. This is a life-threatening condition in which blood volume is severely depleted and the heart reduces its flow of blood through the human body. In the event of an individual entering a state of hypovolemic shock, direct medical attention is required by a doctor who will administer intravenous crystalloids, red blood cell transfusion, platelet transfusion, or blood plasma transfusion. The emergency care lasts until the patient is out of shock and stable again.

Dehydration can also cause and worsen existing urinary tract infections (UTI). UTIs can have serious implications if left untreated, like kidney infections. Preventing dehydration can serve as a mechanism for UTI treatment. Increasing liquid intake along with increased frequency of urination has been shown to prevent UTIs. UTIs are treated with common antibiotics that can be prescribed by urgent care, the emergency room, or a primary care doctor. Serious cases of UTIs may require hospitalization and stronger antibiotics. UTIs can reoccur but are generally an isolated infection incident that is treated the amount of time it takes to run the course of antibiotics.

Cerebral edema is a severe complication of traumatic brain injury (TBI) and also can occur by rehydrating a dehydrated patient too quickly. The brain accumulates fluid and swells, causing significant mortality for TBI patients in particular. The sudden increase in intracranial pressure can affect the brain in relation to respiration and cardiac functioning. This condition must be treated quickly and direct emergency measures must be taken to stop the swelling. If long-term complications arise from damage to the brain, a neurologist can help a patient manage their symptoms.

Another potential complication of dehydration is seizure. Electrolyte imbalance that occurs in severe dehydration can cause seizures through involuntary muscle contractions and unconsciousness. Seizures from dehydration will likely cease by treating the dehydration in the patient. Therefore, treatment may simply consist of rehydrating the patient in the hospital. If the seizure had lasting effects, a neurologist can be seen for symptom management.

In light of the foregoing, monitoring hydration, rehydrating by drinking fluids, and being aware of any medications that can make a patient susceptible to dehydration is vitally important in the cycle of care. For healthy individuals, these tasks are relatively easy to carry out unless they are in hot, dry weather for prolonged periods of time, or engaged in strenuous exercise for an extended period.

Preliminary treatments such as dietary supplements or reactionary treatments such as electrolyte medications that the patient must consume daily for extended periods of time may be prescribed by the doctor for very good reasons: preemptive strategies, long-term management of the condition, and better general health. Patients, however, especially those in the elderly sub-group, often are disillusioned with the idea of having to consume supplements and medications on a strict daily regimen.

Finally, a major conflict of interest exists between doctors and insurance companies. Doctors are principally and wholly concerned with providing the best and most comprehensive treatments to ensure a high quality of life for the patient. Meanwhile insurance companies are often the major limiting factor in the quality or number of treatments that a doctor can perform, thereby limiting the potential for improvement in quality of life for the patient.

There are thus primarily two arenas that exist within the cycle of care for dehydration. The first arena is the “home front,” which encompasses the home residences for young children, adults and nursing homes or group care facilities where a marked proportion of elderly individuals reside. The second arena is “healthcare,” which encompasses physician's offices and hospital settings where individuals turn to receive treatment from doctors and medical professionals.

On the home front, the stakeholders who directly finance the cycle of care are the patients, their families, and their caregivers. The forms of care that these stakeholders finance are almost entirely preventative measures, such as adequate consumption of water, electrolyte drinks and supplements, dietary supplements, and lifestyle changes. Relative to the other costs associated with the home front cycle of care, the costs of ensuring adequate consumption of water and making lifestyle changes are negligible. Regarding electrolyte drinks and packet supplements, the patients and their families bear the costs. Though cheap compared to “healthcare” costs, electrolyte drinks and packet supplements are paid for out-of-pocket by patients and families. These costs are up to their discretion depending on how often and how much they wish to consume to combat dehydration, especially in the unique cases of athletes, individuals who work outdoors, or individuals who have a higher risk of developing dehydration due to medications or pre-existing conditions. In the case of healthcare, the insurance company or the US government typically covers the majority of costs for care and prevention.

### SUMMARY OF THE DISCLOSURE

The disclosure is and includes an apparatus, system and method for providing a hydration detection device. The hydration detection device, includes: a flexible substrate having screen-printed therein an electrical circuit comprising a plurality of layers of conductive and dielectric inks; a sensor upon the flexible substrate, wherein the sensor is capable of detecting element data indicative of characteristics of sweat; untreated chromatographic paper placed so as to contact skin of a user, and capable of wicking the sweat from the skin to the sensor along a micro-sized fluidic channel; and a bluetooth antenna on the flexible substrate and communicative with the sensor, and capable of communicating the element data wirelessly to an application capable of indicating to the user a propriety of the hydration of the user based upon the element data.

### BRIEF DESCRIPTION OF THE FIGURES

This disclosure is illustrated by way of example and not by way of limitation in the accompanying figure(s). The figure(s) may, alone or in combination, illustrate one or more embodiments of the disclosure. Elements illustrated in the figure(s) are not necessarily drawn to scale. Reference labels may be repeated among the figures to indicate corresponding or analogous elements.

FIG. 1 illustrates aspects of an exemplary embodiment of the present invention;

FIG. 2 illustrates aspects of an exemplary embodiment of the present invention;

FIG. 3 illustrates aspects of the embodiments;

FIG. 4 illustrates aspects of the embodiments;

FIG. 5 illustrates aspects of an exemplary embodiment of the present invention;

FIG. 6 illustrates aspects of an exemplary embodiment of the present invention;

FIG. 7 illustrates aspects of the embodiments;

FIG. 8 illustrates aspects of the embodiments;

FIG. 9 illustrates aspects of an exemplary embodiment of the present invention;

FIG. 10 illustrates aspects of an exemplary embodiment of the present invention;

FIG. 11 illustrates aspects of the embodiments;

FIG. 12 illustrates aspects of the embodiments; and

FIG. 13 illustrates aspects of an exemplary embodiment of the present invention;

FIG. 14 illustrates aspects of an exemplary embodiment of the present invention;

FIG. 15 illustrates aspects of an exemplary embodiment of the present invention;

FIG. 16 illustrates aspects of an exemplary embodiment of the present invention;

FIG. 17 illustrates aspects of an exemplary embodiment of the present invention;

FIG. 18 illustrates aspects of an exemplary embodiment of the present invention;

FIG. 19 illustrates aspects of an exemplary embodiment of the present invention;

FIG. 20 illustrates aspects of an exemplary embodiment of the present invention;

FIG. 21 illustrates aspects of an exemplary embodiment of the present invention;

FIG. 22 illustrates aspects of an exemplary embodiment of the present invention;

FIG. 23 illustrates aspects of an exemplary embodiment of the present invention;

FIG. 24 illustrates aspects of an exemplary embodiment of the present invention;

FIG. 25 illustrates aspects of an exemplary embodiment of the present invention;

FIG. 26 illustrates aspects of an exemplary embodiment of the present invention;

FIG. 27 illustrates aspects of an exemplary embodiment of the present invention;

FIG. 28 illustrates aspects of an exemplary embodiment of the present invention;

FIG. 29 illustrates aspects of an exemplary embodiment of the present invention;

FIG. 30 illustrates aspects of an exemplary embodiment of the present invention;

FIG. 31 illustrates aspects of an exemplary embodiment of the present invention;

FIG. 32 illustrates aspects of an exemplary embodiment of the present invention;

FIG. 33 illustrates aspects of an exemplary embodiment of the present invention;

FIG. 34 illustrates aspects of an exemplary embodiment of the present invention;

FIG. 35 illustrates aspects of an exemplary embodiment of the present invention;

FIG. 36 illustrates aspects of an exemplary embodiment of the present invention;

FIG. 37 illustrates aspects of an exemplary embodiment of the present invention;

FIG. 38 illustrates aspects of an exemplary embodiment of the present invention;

FIG. 39 illustrates aspects of an exemplary embodiment of the present invention;

FIG. 40 illustrates aspects of an exemplary embodiment of the present invention;

FIG. 41 illustrates aspects of an exemplary embodiment of the present invention;

FIG. 42 illustrates aspects of an exemplary embodiment of the present invention;

FIG. 43 illustrates aspects of an exemplary embodiment of the present invention;

FIG. 44 illustrates aspects of an exemplary embodiment of the present invention;

FIG. 45 illustrates aspects of an exemplary embodiment of the present invention;

FIG. 46 illustrates aspects of an exemplary embodiment of the present invention;

FIG. 47 illustrates aspects of an exemplary embodiment of the present invention;

FIG. 48 illustrates aspects of an exemplary embodiment of the present invention;

FIG. 49 illustrates aspects of an exemplary embodiment of the present invention;

FIG. 50 illustrates aspects of an exemplary embodiment of the present invention;

FIG. 51 illustrates aspects of an exemplary embodiment of the present invention;

FIG. 52 illustrates aspects of an exemplary embodiment of the present invention;

FIG. 53 illustrates aspects of an exemplary embodiment of the present invention;

FIG. 54 illustrates aspects of an exemplary embodiment of the present invention;

FIG. 55 illustrates aspects of an exemplary embodiment of the present invention;

FIG. 56 illustrates aspects of an exemplary embodiment of the present invention;

FIG. 57 illustrates aspects of an exemplary embodiment of the present invention;

FIG. 58 illustrates aspects of an exemplary embodiment of the present invention;

FIG. 59 illustrates aspects of an exemplary embodiment of the present invention;

FIG. 60 illustrates aspects of an exemplary embodiment of the present invention;

FIG. 61 illustrates aspects of an exemplary embodiment of the present invention;

FIG. 62 illustrates aspects of an exemplary embodiment of the present invention;

FIG. 63 illustrates aspects of an exemplary embodiment of the present invention;

FIG. 64 illustrates aspects of an exemplary embodiment of the present invention;

FIG. 65 illustrates aspects of an exemplary embodiment of the present invention;

FIG. 66 illustrates aspects of an exemplary embodiment of the present invention;

FIG. 67 illustrates aspects of an exemplary embodiment of the present invention;

FIG. 68 illustrates aspects of an exemplary embodiment of the present invention;

FIG. 69 illustrates aspects of an exemplary embodiment of the present invention;

FIG. 70 illustrates aspects of an exemplary embodiment of the present invention;

FIG. 71 illustrates aspects of an exemplary embodiment of the present invention;

FIG. 72 illustrates aspects of an exemplary embodiment of the present invention; and

FIG. 73 illustrates aspects of an exemplary embodiment of the present invention.

### DETAILED DESCRIPTION

The figures and descriptions provided herein may have been simplified to illustrate aspects that are relevant for a clear understanding of the herein described apparatuses, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are known in the art, and because they do not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

Embodiments are provided throughout so that this disclosure is sufficiently thorough and fully conveys the scope of the disclosed embodiments to those who are skilled in the art. Numerous specific details are set forth, such as examples of specific components, devices, and methods, to provide a thorough understanding of embodiments of the present disclosure. Nevertheless, it will be apparent to those skilled in the art that certain specific disclosed details need not be employed, and that embodiments may be embodied in different forms. As such, the embodiments should not be construed to limit the scope of the disclosure. As referenced above, in some embodiments, well-known processes, well-known device structures, and well-known technologies may not be described in detail.

The terminology used herein is for the purpose of describing particular embodiments only and is not intended to be limiting. For example, as used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The steps, processes, and operations described herein are not to be construed as necessarily requiring their respective performance in the particular order discussed or illustrated, unless specifically identified as a preferred or required order of performance. Itis also to be understood that additional or alternative steps may be employed, in place of or in conjunction with the disclosed aspects.

When an element or layer is referred to as being “on”, “engaged to”, “connected to” or “coupled to” another element or layer, it may be directly on, engaged, connected or coupled to the other element or layer, or intervening elements or layers may be present, unless clearly indicated otherwise. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). Further, as used herein the term “and/or” includes any and all combinations of one or more of the associated listed items.

Yet further, although the terms first, second, third, etc. may be used herein to describe various elements, components, regions, layers and/or sections, these elements, components, regions, layers and/or sections should not be limited by these terms. These terms may be only used to distinguish one element, component, region, layer or section from another element, component, region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the embodiments.

Processor-implemented control modules, systems and method may be disclosed herein that may provide access to and transformation of a plurality of types of digital content, including but not limited to video, image, text, audio, metadata, algorithms, identifiers, interactive and document content, and which track, deliver, manipulate, transform, transceive and report the accessed content to control and execute the manufacturing processes discussed herein. Described embodiments of these control modules, systems and methods processed by a processing system are intended to be exemplary and not limiting.

A major issue with preventing dehydration has been finding a way for individuals to monitor their hydration levels outside of clinical settings. To remedy this issue, companies have developed affordable patches that monitor levels through sweat. Not only do these patches provide personal hydration monitoring, but they are also financially advantageous as they reduce dehydration-related hospitalizations which cause significant financial strains.

Another existing solution is the jelly drop, which is a water-infused treat that was devised for hydration. Although the specific target audience for jelly drops was initially the elderly, these are consumed by people of all ages as a preventative measure or treatment for mild cases of dehydration.

Electrolytic drinks, such as Gatorade, are another preventative measure and treatment for mild dehydration. These drinks were originally designed for athletes to ensure hydration and increase on-field performance. Now they are also used by the general population and pose as treatment options for mild cases of dehydration.

When a patient doesn't seek treatment in its mild state, dehydration can become severe, which requires more extreme measures, such as IV therapy. While such treatment options are effective, the issue that emerges is in regard to affordability. These treatment options mainly require hospitalization and administration from health care professionals, which is usually costly.

Thus emerges the need to create affordable, at-home preventative measures and treatments.

The lack of accuracy with the aforementioned monitoring devices presents a most significant issue. Many existing devices analyze biomarkers in sweat to correlate levels of total-body fluid volume to quantify fluid loss. Research shows that the correlations between sweat and total-body fluid volume are weak. Thus, some devices monitor hydration levels in real-time while also collecting other information such as heart-rate, activity tracking, and sleep tracking, to generate hydration recommendations. However, such devices typically carry a significant cost.

Another limitation with sweat analyzing devices is that enough sweat must be collected for accurate readings. The need to exercise to generate sweat limits the use of the patch to active individuals, which is problematic because it removes a large population of those that are more susceptible to becoming dehydrated.

Many existing products are also bulky and impractical for everyday use. This is especially an issue for athletes because bulky devices that are strapped to certain parts of the body can reduce range of motion and require constant adjustment.

The needs for monitoring devices to prevent dehydration thus focus on accuracy of the device, convenience, and ease of use. This favors a quick and easy solution while maintaining the highest standard of accuracy in patient care.

Thus, embodiments provide a way to monitor electrolyte imbalance to prevent dehydration and related conditions. The embodiments provide: a biosensor and analysis on-site, such as many be subsequently transmitted to an app via Bluetooth; a portable and simple to use hydration monitor that senses sweat by monitoring just a single analyte; a device that may be screen-printed via mesh screens; a device that is small in size, such as 2 cm by 3 cm; a device that may be cheap to manufacture (such as under $5); a device with the ability to operate by collecting just small amounts of sweat (such as −20 μL).

Further, the embodiments may provide a baseline setup in-app to determine normal levels customized to each individual user. The device may sense sweat using multiple analytes, rather than a single analyte. The device may be reusable (i.e., the device may include disposable components and multi-use circuitry components). The device may be aesthetically pleasing and have a comfortably wearable design. Further, the device may include the capacity to induce sweat by thermal or electrical means.

The embodiments may take the form of an adhesive disposable patch with an adjacent circuit that is reusable. The patch utilizes paper microfluidics to collect microliters of sweat, which is analyzed using a printed OECT (Organic Electrochemical Transistor) biosensor that detects sodium ions. The reusable circuit may power, process, and transmit the data via, for example, Bluetooth Low Energy (BLE) to a mobile app, which will provide information on hydration levels. The patch design is applicable to the entire population and holds significant potential in the fitness tracking market relevant to athletes. “Athletes” is used to refer to individuals who are interested in or participate in physical activity for exercise.

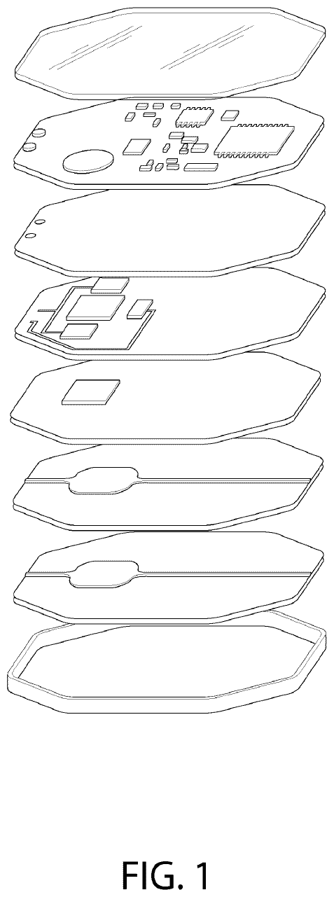

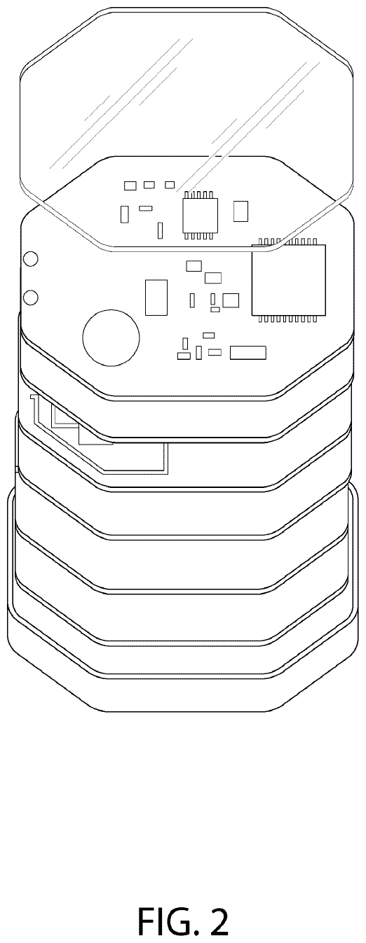

In a first embodiment, illustrated in FIG. 1, the first layer consists of adhesive strips that allow for the patch to be fixed onto the skin. Layers 2 and 3 form the paper microfluidics channel for which the bottom is hydrophilic and consists of an inlet with the top layer being hydrophobic to direct sweat a certain direction. The next layer is an insulative layer that protects the PEDOT:PSS layer above from the sweat. The PEDOT:PSS layer consists of a source, drain and gate electrode with wires, such as silver wires, that lead to the edge of the patch. The sixth layer is a conductive layer that connects the silver wires from the layer below to the circuit layer above. This circuit layer is constructed based off a reference circuit design that transmits data from the biosensor to an app through Bluetooth. The final layer is a PDMS encasement layer that protects the circuit layer. Exemplary dimensions of the patch are shown in FIG. 2. The device functions by collecting sweat through the paper microfluidics, and the OECT biosensor analyzes the sodium concentration. This information is relayed through a battery powered BLE circuit and connected to a mobile phone app that provides hydration information. The entire device is disposable.

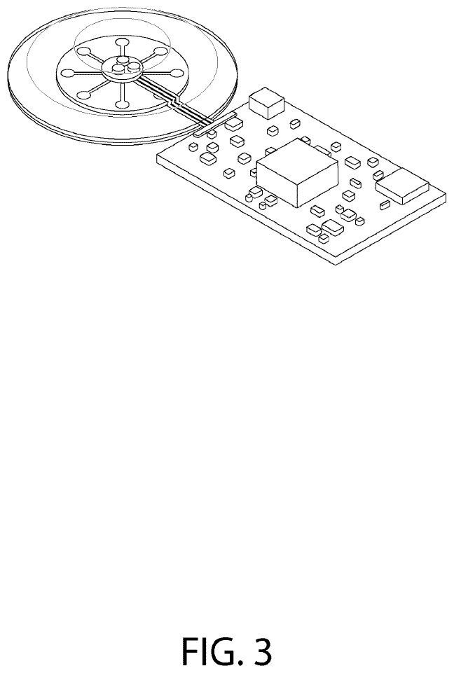

FIG. 3 illustrates a second embodiment. This is a two-part device that consists of a patch and a circuit which are connected with silver wiring. The first layer of the patch is the paper microfluidics with the grey representing the converging hydrophilic inlets and the yellow representing the hydrophobic barrier. The area below the purple circle is a central reservoir where the sweat collects from each of the inlets. The second layer is denoted with a purple circle which is the PEDOT:PSS. Layer 3 contains the carbon ink source, drain and gate electrodes which are connected with silver wires that lead to the circuit. The final layer is the adhesive/protectant layer that encases the biosensor.

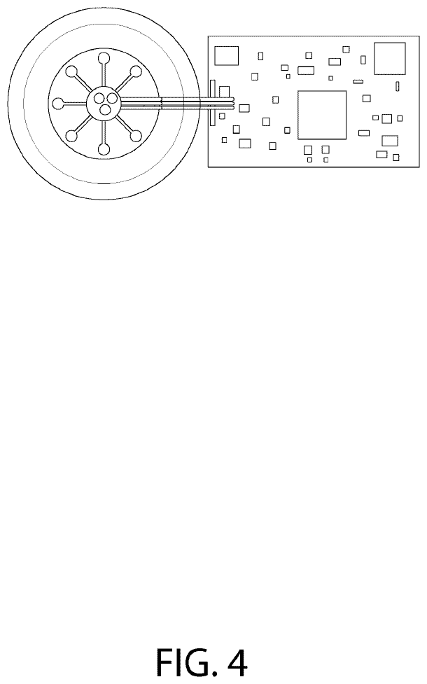

Exemplary dimensions of the design of FIG. 3 are shown in FIG. 4. The device functions by collecting sweat through multiple inlets in the paper microfluidics. The OECT biosensor analyzes the sodium concentration. This information is relayed through a connector onto a battery powered BLE circuit and connected to a mobile phone app that provides hydration information. The patch component is disposable, and the circuit is reusable.

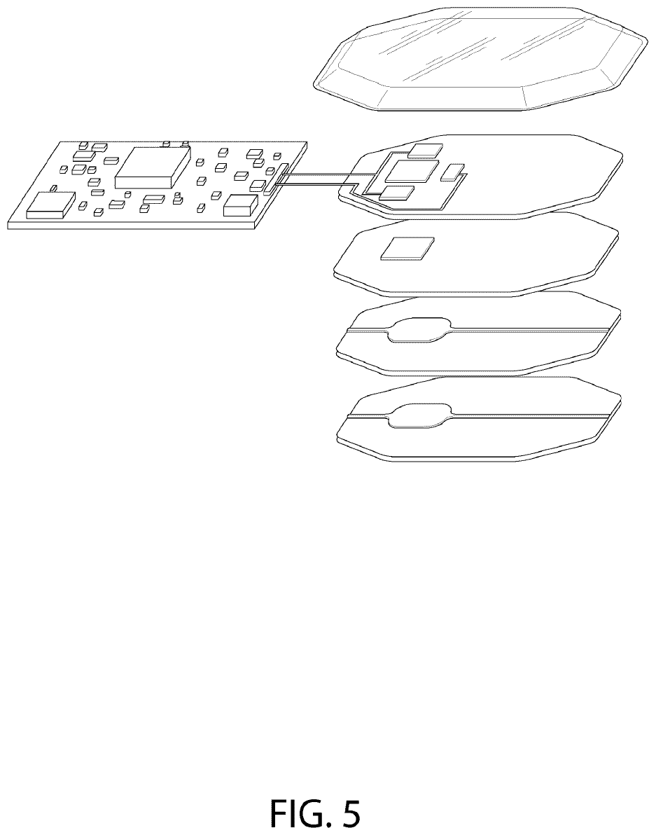

An additional embodiment is shown in FIG. 5. Like the previous designs, this solution has the circuit separate from the biosensor as it simplifies the detachability aspect to make the patch reusable. Layers 1 and 2 form the paper microfluidic channels with the inlet, electroactive area and outlet. The layer above is the insulative layer that separates the liquid electrolyte in the paper from the biosensor. Layer 4 is the PEDOT:PSS layer which contains the carbon ink source, drain and gate electrodes which are connected with silver wires. The final layer is the adhesive/protectant layer that encases the patch.

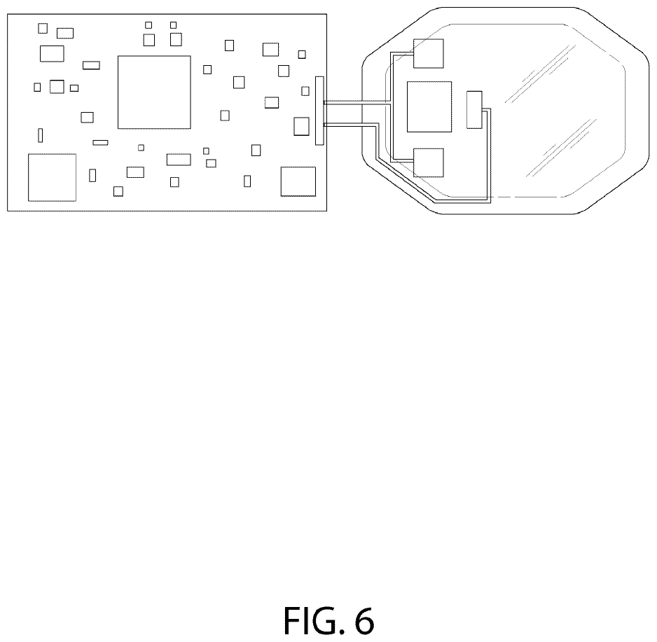

Exemplary dimensions of the design of FIG. 5 are shown in FIG. 6. The device functions by collecting sweat through an inlet in the paper microfluidics. The OECT biosensor analyzes the sodium concentration. This information is relayed through a connector onto a battery powered BLE circuit and connected to a mobile phone app that provides hydration information. The patch component is disposable, and the circuit is reusable.

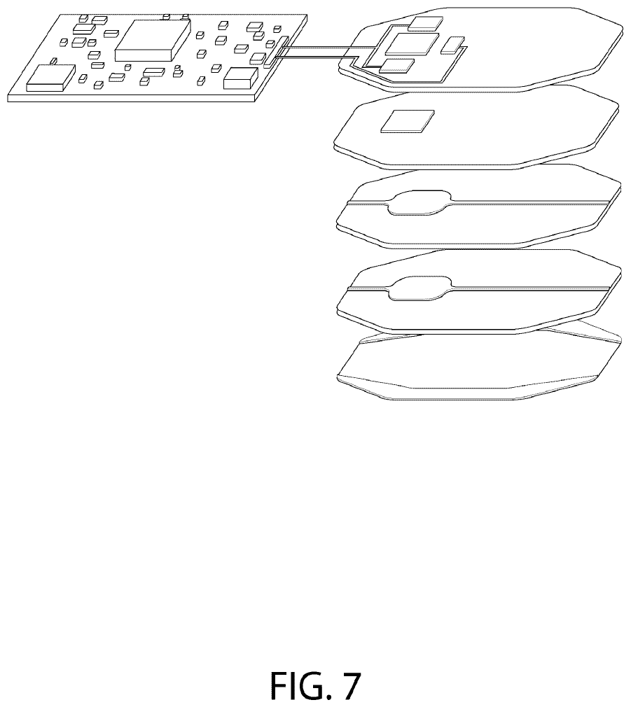

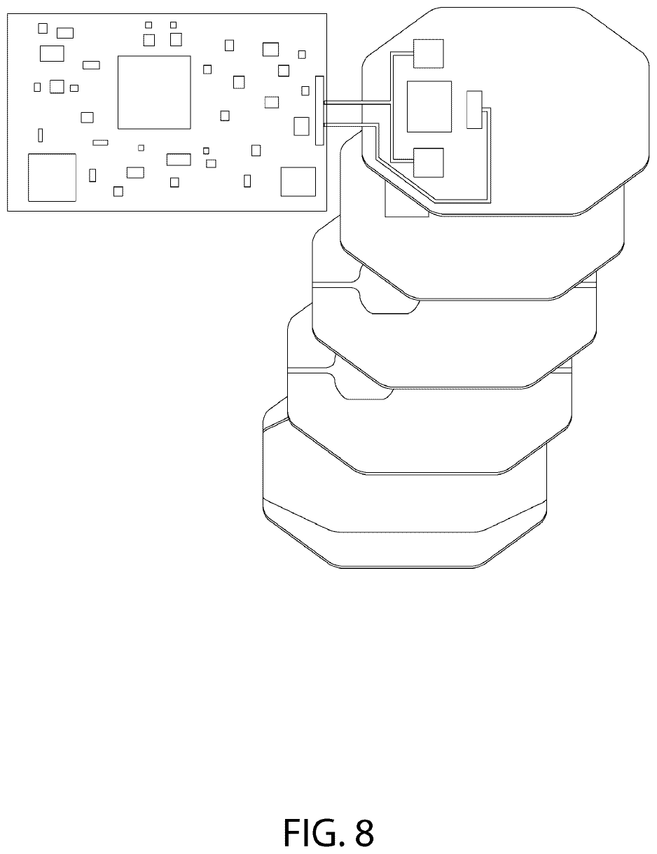

An additional embodiment, shown in FIG. 7, comprises a combination of the first and third designs. From the first design, the adhesive concept where it was used as a bottom layer was substituted for the encapsulant adhesive layer of the third design. All else with the final design is similar to design 3, for which the dimensions are shown in FIG. 8. The device functions by collecting sweat through multiple inlets in the paper microfluidics. The OECT biosensor analyzes the sodium concentration. This information is relayed through a connector onto a battery powered BLE circuit and connected to a mobile phone app that provides hydration information. The patch component is disposable, and the circuit is reusable.

Table 1 illustrates the relevant standards for the embodiments.

TABLE 1

| Standard Name | Specific Standard Code and Description |
|---|---|
| Sweat Testing | Uses standard ISO105-E04-2008 to develop |
| | synthetic sweat of NaCl and two other |
| | components into DI water. |
| Electrostatic | Uses standard ANSI/ESD S20.20 to ensure |
| Discharge | process performance, control products, decrease |
| | failure rate, minimize rework, and reduce |
| | expenses. |
| Environmental | Jabil maintains ISO 14001 Environmental |
| Management | Management System (EMS) certification to |
| | minimize how operations negatively affect he |
| | environment |
| Health and Safety | Jabil maintain OHSAS 18001 Occupational |
| | Health and Safety certification to control risks |
| | and hazards. |
| Wellness or | IEC/UL 62368-1 focuses on identifying |
| Non-Medical | possible injuries caused by hazards and requires |
| Wearable | safeguards for all components and subsystems. |
| Medical Device | IED 60601-1-11 applies to the basic safety and |
| Safety | essential performance of home use medical |
| | devices (devices intended for users in a |
| | non-clinical or transitory setting) |
| Biocompatibility | ISO 10993 defines requirements for |
| | biocompatibility of medical devices to manage |
| | biological risk |
| Communication | ISO 11073 directs the communication of |
| | medical data for point-of-care medical devices. |
| Electromagnetic | IED 60601-2 defines testing and safety limits |
| Compatibility | for medical devices that communicate via |
| | Bluetooth or any other RF transmitters. |
| Usability | IED 60601-6 specifies a process for |
| | manufacturers to analyze and validate usability |
| | as it relates to basic safety. |
| Design Control | ISO 13485 provides guidance on design control |
| | for medical devices, including input |
| | requirements and performance verification. |
| Structural Testing | ISO 14971 impacts design and testing to verify |
| | that a wearable sensor is free of structural |
| | defects that could pose a risk for users or |
| | compromise proper functioning. |

The microfluidic portion of the patch may be created using multiple steps. The first step in fabricating may be creating a channel; chromatography paper may be selected as the material due to its hydrophilic properties. That is, paper having chromatographic properties provides wicking capabilities, which will very efficiently wick sweat from the skin to the sensor. Moreover, untreated chromatographic paper maintains its wicking capability across a large number of uses. Additionally or alternatively, a hydrophobic barrier may be provided using a stencil from a polyethylene terephthalate (PET) sheet.

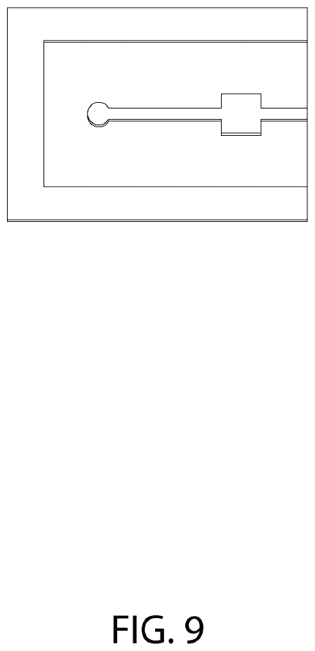

In FIG. 9, the center portion is the microfluidic channel, and the sheet on which the channel resides is the PET substrate. Note that the square portion of the channel is exposed and will allow the fluid to seep up from below and become exposed to the PEDOT:PSS transistor channel of the OECT biosensor.

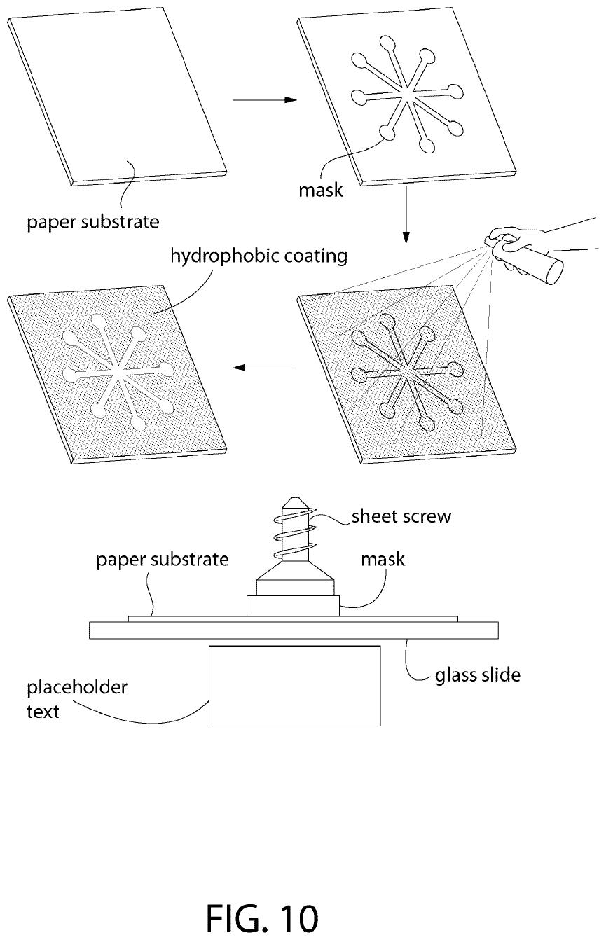

The microfluidic portion of the patch may be formed by laying a PET stencil on chromatography paper and using a spray with hydrophobic properties to pattern a defined channel onto the paper. The spray with hydrophobic properties may be a water repellent coating that contains organic solvent, such as including acetone, xylene, or liquefied petroleum gas. The PET thickness may be between, for example, about 7 micrometers to 20 micrometers, and more preferably 10 micrometers. This is summarized in FIG. 10.

Two main functionalities of the embodiments are: the wicking of liquid by and from the microfluidic channel; and the adhesive properties. As indicated, the microfluidics may be formed of paper. The adhesive may both attach the channel to the PET substrate and the entire patch to the wearer's skin. Further included may be an OECT biosensor, a wireless readout device, and a mobile app platform connected to the device via a Bluetooth Low Energy connection. The channel's electroactive area may be a square, a diamond shape, and/or a shape with rounded corners, as such a geometry promotes more efficient collection of fluid for lateral flow.

Screen-printing is a mass-production process that is employed in some embodiments. The biosensor may be fabricated using this method, such as comprising a screen printed flexible hybrid electronic. The screen-printing may use custom screens. The wire traces may be formed of silver; carbon may be used on the source, drain, and gate electrodes; PEDOT:PSS may serve as the organic channel; and dielectric/insulation may be used to encase everything besides the electroactive area.

Additionally, the bio-sensor may be or be connected to a PCB. A female and male connector may attach and detach the biosensor from the PCB. The connection of the disposable patch to the reusable circuit and microcontroller may be a standard 3-prong connector. The patch, including the biosensor and the paper microfluidics, is designed to be disposable.

The patch may adhere to the skin, or may simply attach with Velcro and held on by the armband. A simple Velcro armband with a pocket for the PCB allow for easy patch removal and replacement. That is, a velcro pad may secure the armband.

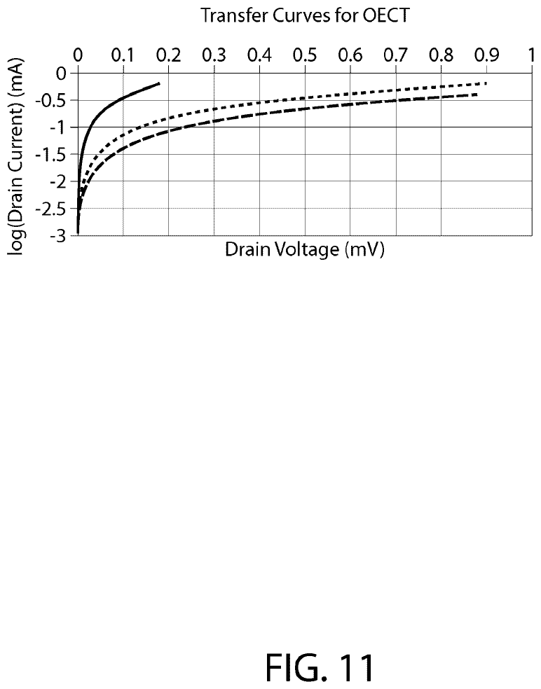

The embodiments incorporate the ability to wick small amounts of sweat (−15 μL) using the patterned chromatography paper. The functioning OECT biosensor is able to read drain current data in microamperes while running a chronoamperometry program using an interconnected app. A voltage is placed on the gate and provided a stepping voltage to generate distinct transfer curves; finally, common ground was placed on the sensor transistor source. The transfer curves follow similarly to the expected trends. The results display changes in the drain current at different stepping gate voltages, which indicates sensor functionality. These transfer curves are shown in FIG. 11.

Ten solutions prepared from PBS and NaCl crystals to encompass ranges from 0 (PBS blank) to 10−8 M were provided. Each solution wicked to the electroactive region of the disposable patch in 20 μL droplets allows the app to run a chronoamperometry program and to display the drain current results on a graphical user interface (GUI).

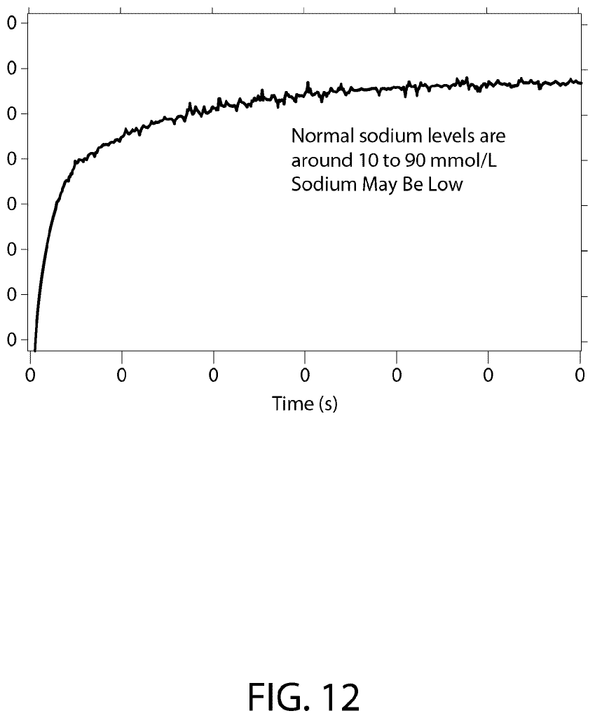

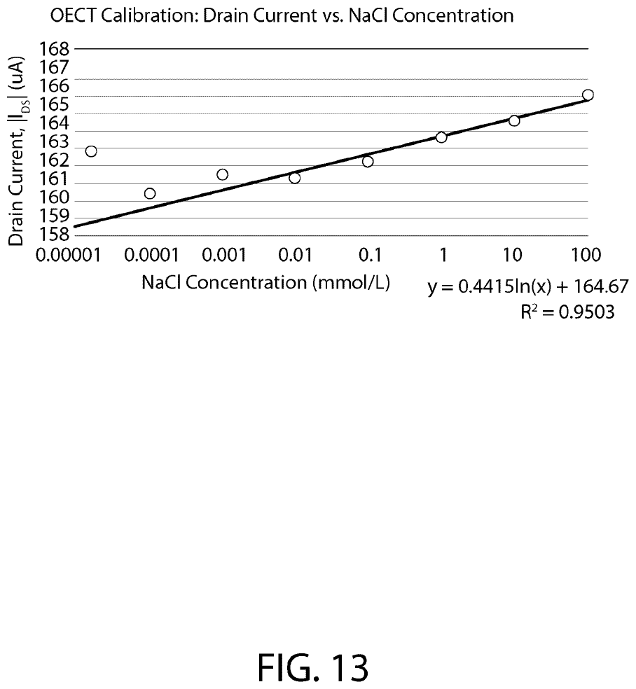

This can be done because a linear relationship exists between the solution NaCl molarity and the drain current. The GUI in this example may indicate that “Sodium may be low,” “Sodium may be high,” or “No sodium abnormalities,” as indicated in FIG. 12. The sensor calibration may be performed as in FIG. 13.

Thus, paper microfluidics may be used to properly produced defined, accurate channels that wick NaCl solutions, i.e., sweat, directly to the electroactive region of a biosensor. The biosensor may be a screen-printed OECT, which may yield consistent current outputs for chronoamperometry at different NaCl concentrations.

The BLE circuitry operates, reads, and transmits the OECT data to a mobile app. The app code is used to analyze sodium concentration and provide that and other readouts at the user's request. Thus, the embodiments provide a device that measures statistical differences in micromolar-scale NaCl solutions to produce a corresponding app output.

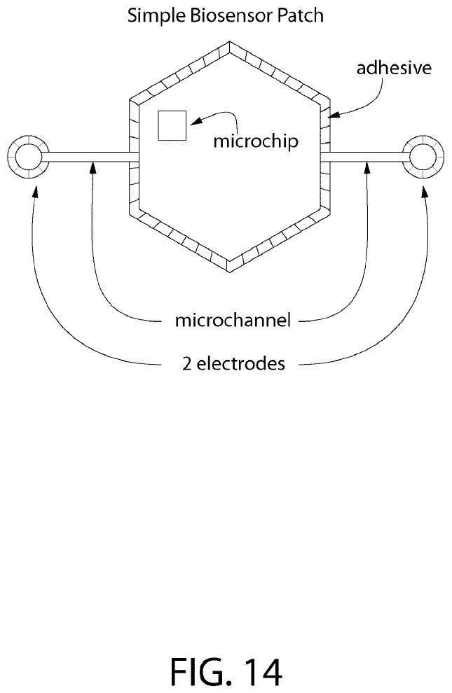

Referring now to FIG. 14, shown is a simple NaCl monitoring patch that contains 2 electrodes. It incorporates a chip and attaches to the skin by an adhesive layer that runs along the edge of the design.

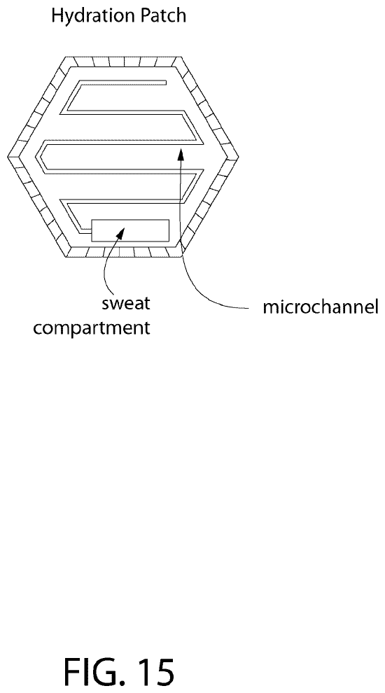

Referring now to FIG. 15, shown is a patch that incorporates microfluidic channels and has a compartment at the bottom for sweat accumulation. Accumulation of sweat is important for accurate analysis of Na⁺ levels.

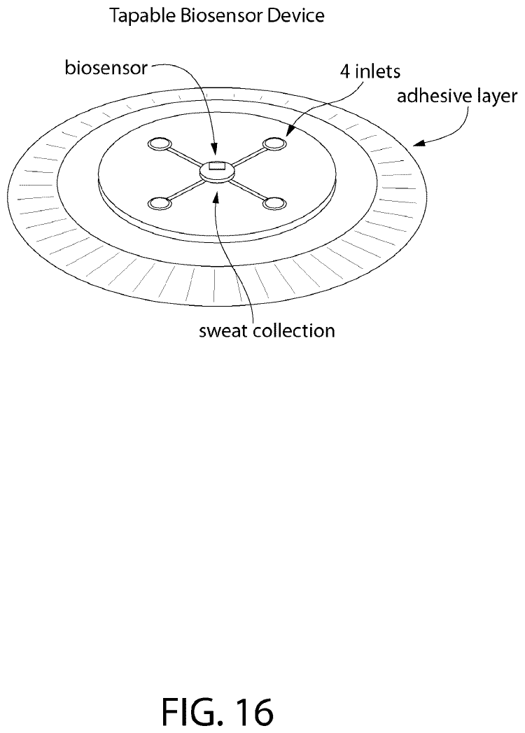

Referring now to FIG. 16, shown is a device that senses hydration levels using four inlets for sweat and then the sweat accumulates in the middle circle. This sensor is then encapsulated with a top adhesive layer that is pressed onto the skin for adherence.

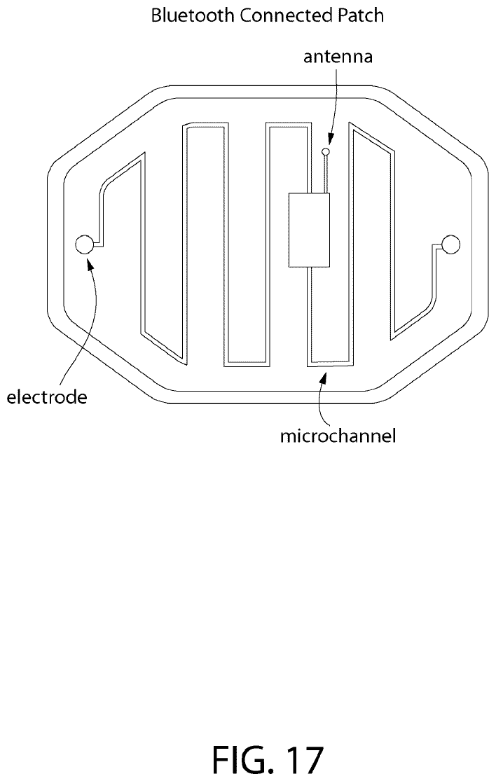

Referring now to FIG. 17, shown is a patch that incorporates 2 electrodes with microfluidic channels. It contains a biosensor to analyze sweat and read out hydration levels to an app via bluetooth for each user-accessibility.

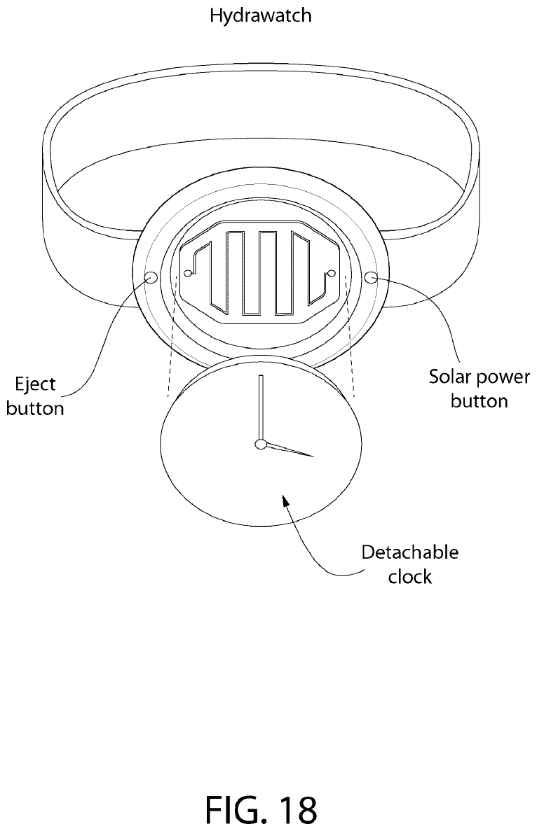

Referring now to FIG. 18, shown is a watch with an inner compartment for the biosensor which is hidden by a detachable clock face. It has a button that induces sweat through heat that is produced using solar power.

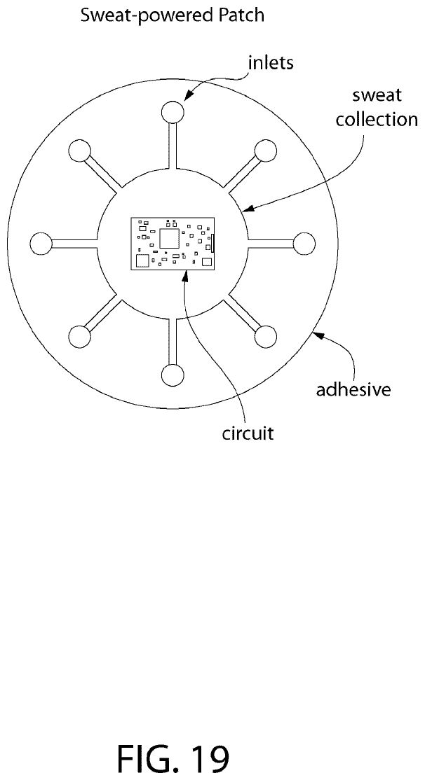

Referring now to FIG. 19, shown is a patch design. With this patch design, there are eight inlets that allow uptake of sweat with a compartment in the middle for the sweat to collect. This sweat is then used to power the biosensor and circuit above which eliminates the need for a battery.

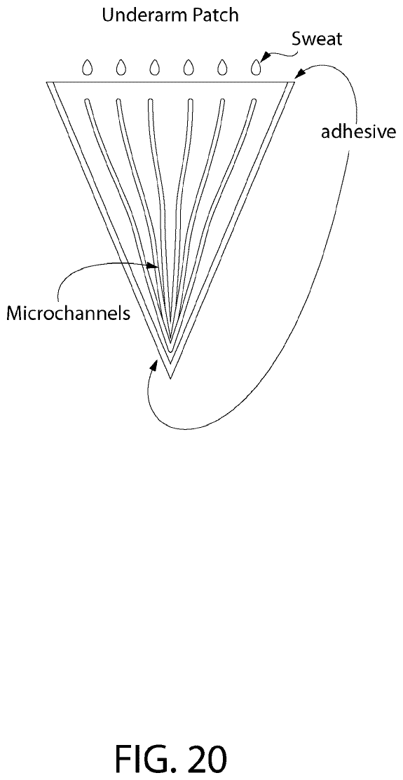

Referring now to FIG. 20, shown is a patch designed specifically for the underarm since it is an area of the body that produces the most sweat. The microchannels have a downward orientation so that gravity can help sweat flow through the patch.

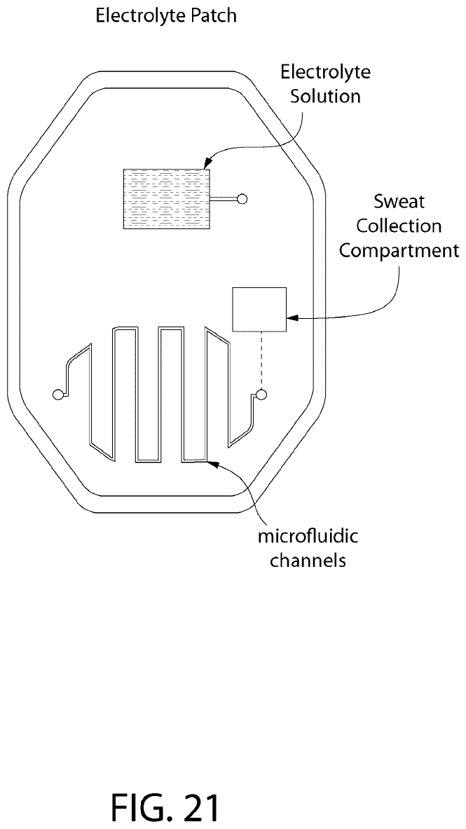

Referring now to FIG. 21, shown is a patch that has a replaceable component that holds an electrolyte solution that is administered when hydration levels are below the acceptable range. There is also a sweat collection compartment that can be ejected when it becomes full.

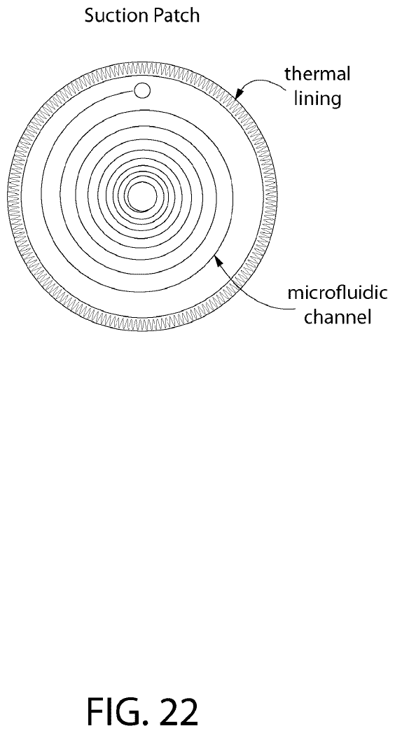

Referring now to FIG. 22, shown is a patch that contains thermal lining as well as suction capabilities which helps induce sweat. The microfluidic channel has a spiral orientation.

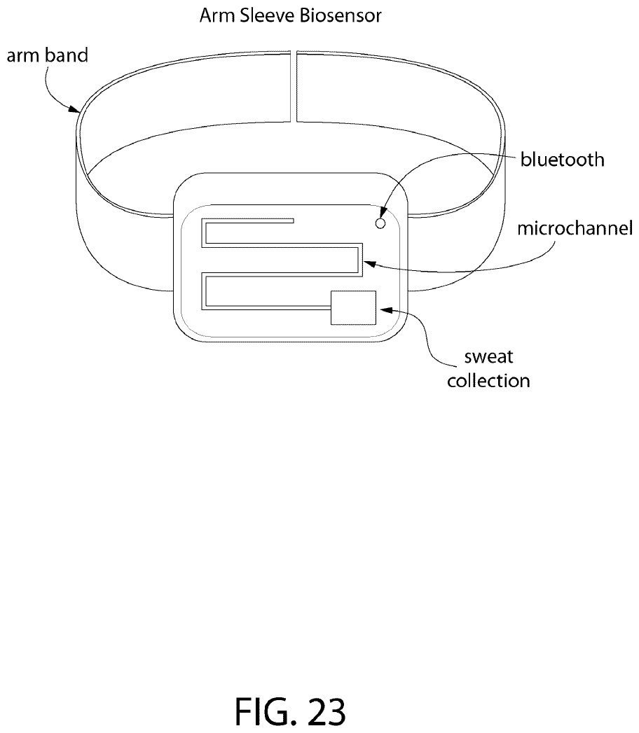

Referring now to FIG. 23, shown is an arm sleeve that can easily be slipped on before a workout to monitor hydration levels. The design also incorporates a bluetooth connection so that data can be accessed via mobile app.

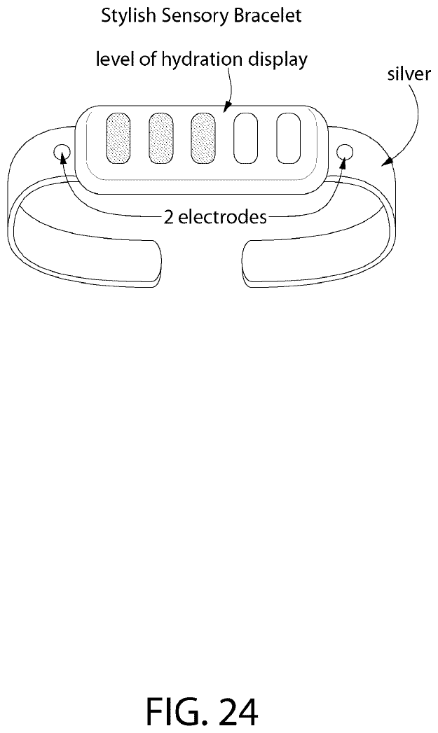

Referring now to FIG. 24, shown is a bracelet that is made of silver and incorporates cooling technology that activates when hydration levels are low. There is a display that shows real time hydration levels for users.

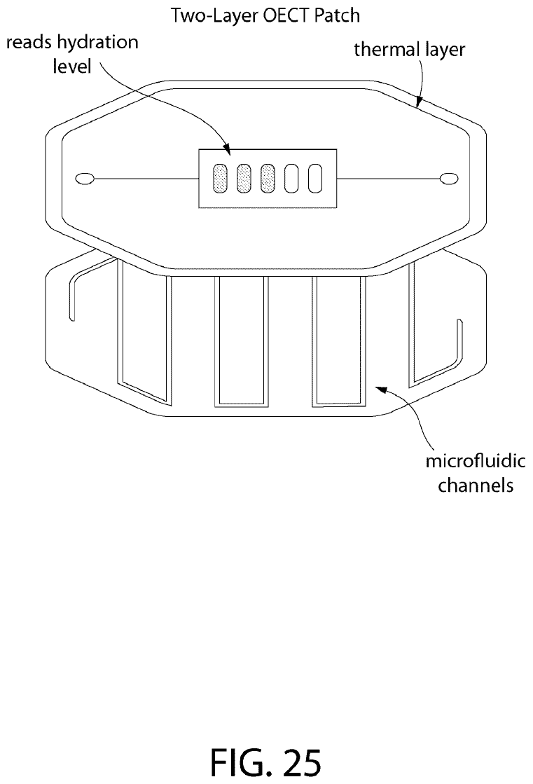

Referring now to FIG. 25, shown is a 2 layer patch design that uses a OECT. It has a thermal layer for inducing sweat and a display that shows level of hydration. The bottom layer contains microfluidic channels.

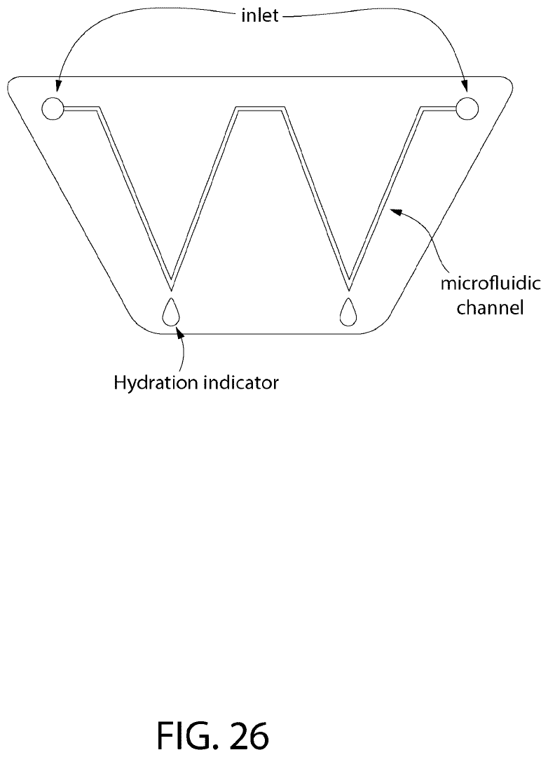

Referring now to FIG. 26, shown is a patch that was inspired by Halloween and has two inlets that flow in a vampire teeth-like orientation. The bottom droplets turn red when the hydration levels are low.

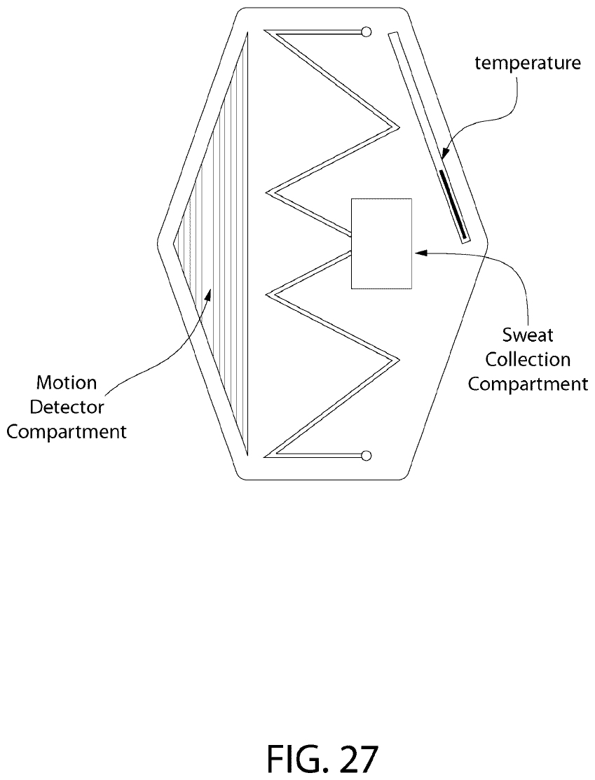

Referring now to FIG. 27, shown is a device that starts monitoring hydration levels with movement. It contains a temperature reader as it is a factor that can impact sweat rate. There is also a sweat collection compartment that is detachable.

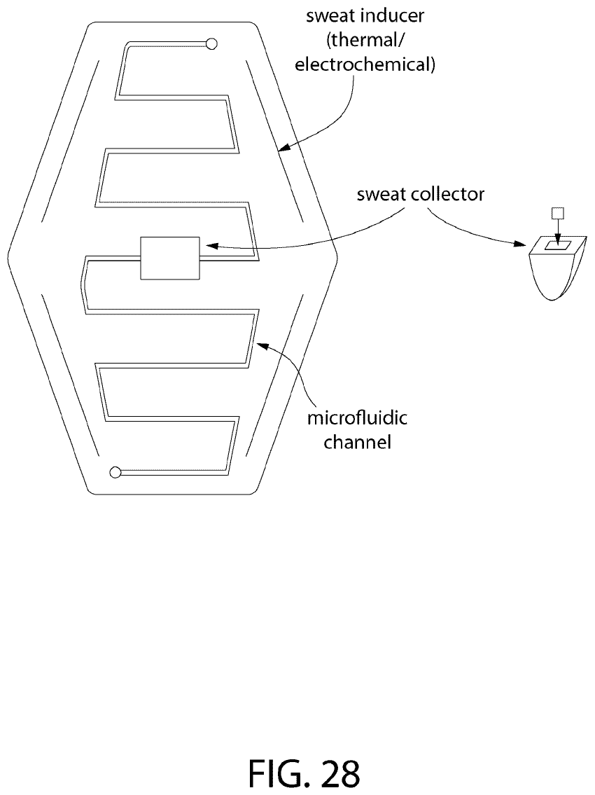

Referring now to FIG. 28, shown is a two piece device that works in unison to deliver hydration levels to users. The patch collects sweat in a compartment that can be detached and put into a second portable device that analyzes hydration levels.

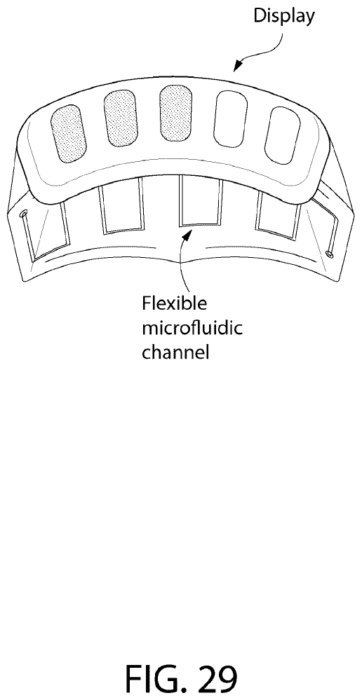

Referring now to FIG. 29, shown is a bendable patch that is slightly dome-shaped and can be pushed onto the skin in a way that it applies a little suction pressure to induce the collection of sweat. It also has a flexible display on top that reads the hydration level.

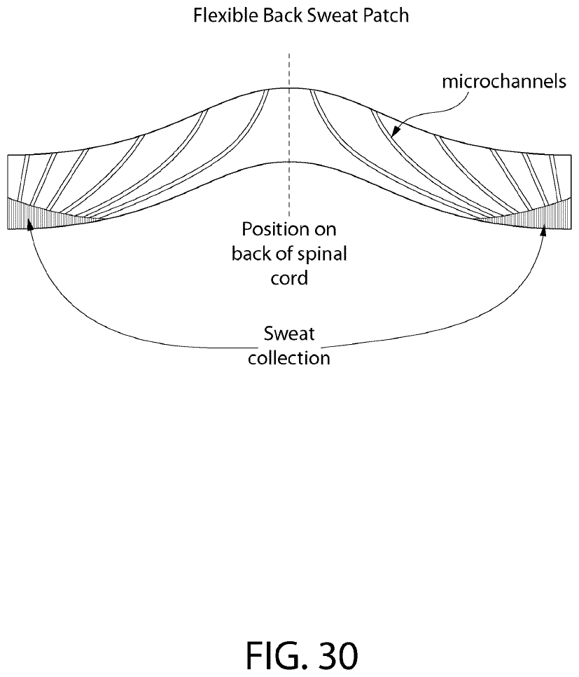

Referring now to FIG. 30, shown is a patch that was designed to collect sweat from the back which is an area where people sweat considerably. There are multiple microchannels that lead downwards to enforce gravity on the sweat so that it collects in the corner areas for accurate analysis.

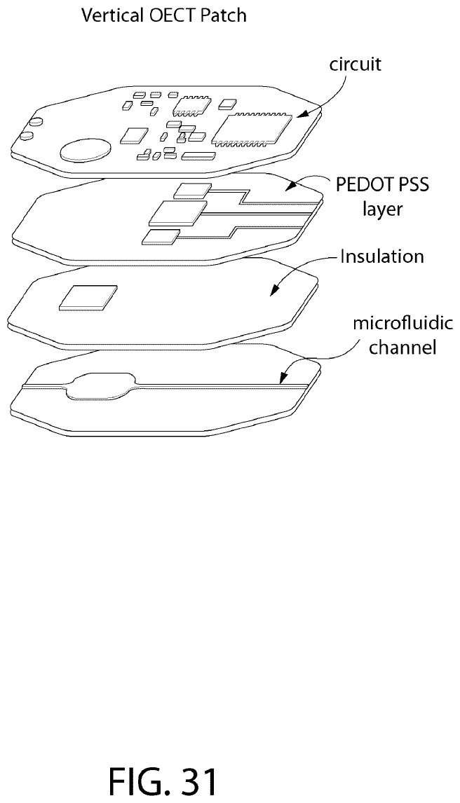

Referring now to FIG. 31, shown is a patch that is an all-inclusive OECT patch that has an adhesive bottom layer and flexible circuit on top. Aside from that this patch is composed of three layers which includes the microfluidic channel, insulation and PEDOT:PSS. The circuit layer is encases with a protectant layer so that is can handle various environmental stressors.

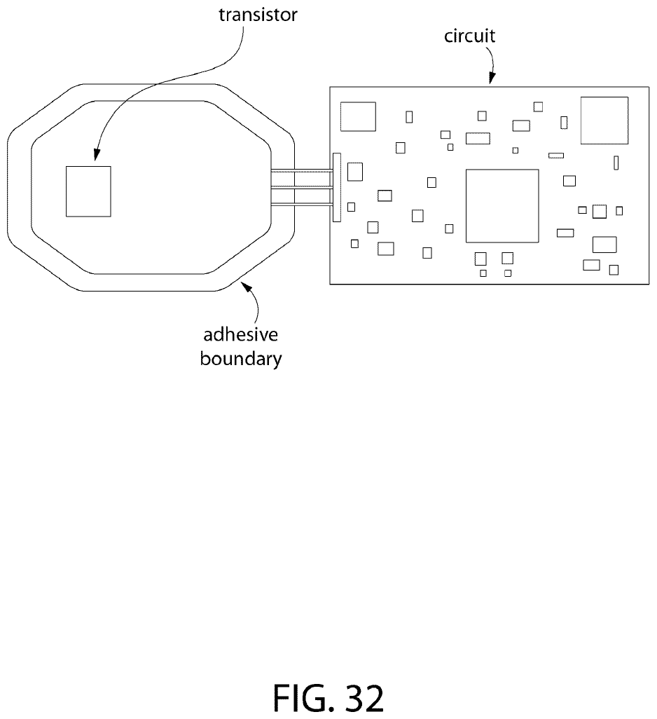

Referring now to FIG. 32, shown is a design that is a two-piece to allow for easier detachability of the disposable patch. It incorporates a regular transistor which then sends the signals to a flexible circuit to process the data and relay information to an app.

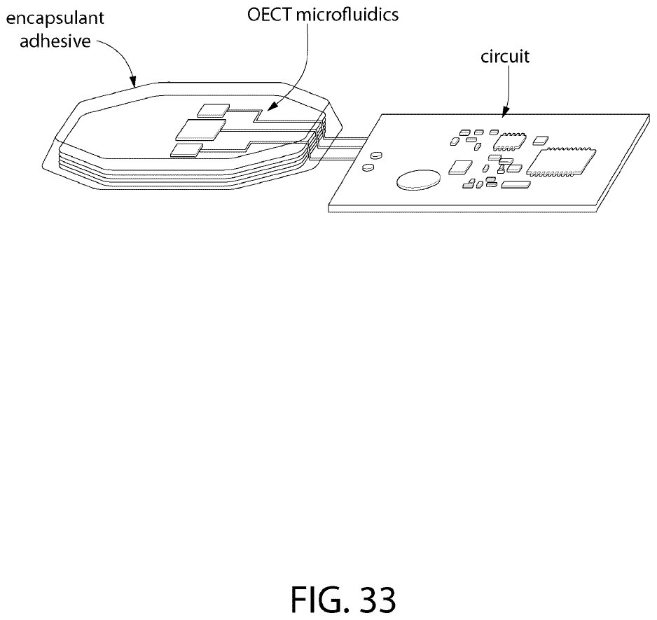

Referring now to FIG. 33, shown is a patch that uses an organic electro chemical transistor and incorporates an encapsulant layer to protect and ahead the patch onto skin. The signals are sent to a separate circuit which also powers the transistor layer.

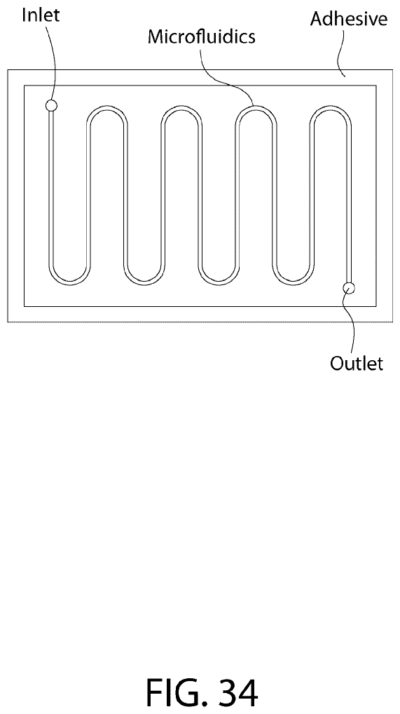

Referring now to FIG. 34, shown is a patch design. Sweat is collected through the inlet, passes through microfluidic channels, and is removed via the outlet. The adhesive patch also includes circuitry in the back including a general ISE biosensor and a BLE connector to an app.

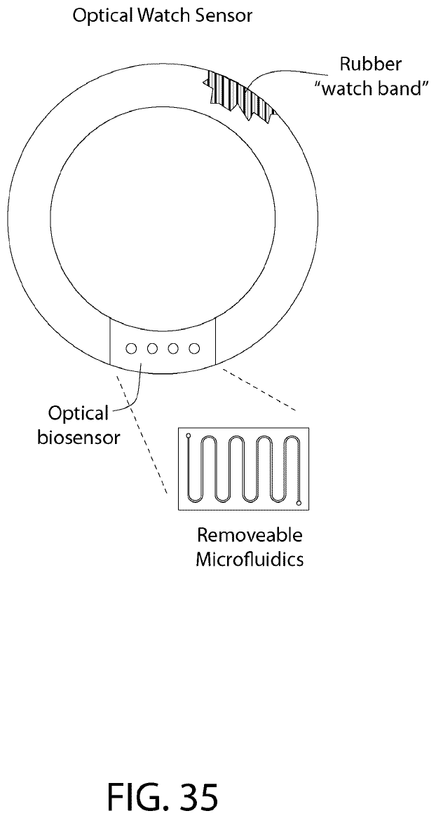

Referring now to FIG. 35, shown is a watch design where sweat is collected via microfluidics and is scanned with an optical biosensor. The watch contains circuitry and a BLE connector to an app. The watch is reusable and has a removeable microfluidics component.

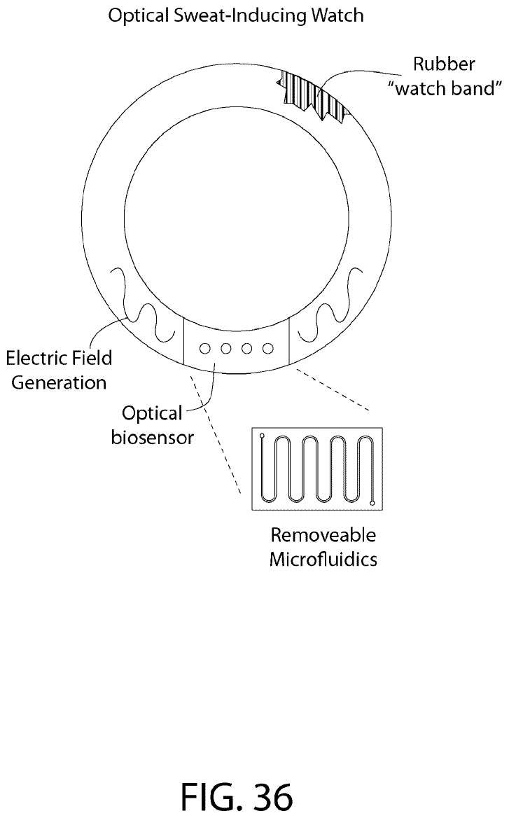

Referring now to FIG. 36, shown is a watch design where sweat is collected via microfluidics and is scanned with an optical biosensor. The watch contains circuitry and a BLE connector to an app. The watch is reusable and has a removeable microfluidics component. This modification adds an electric field generation which induces sweat artificially to collect a sample easier.

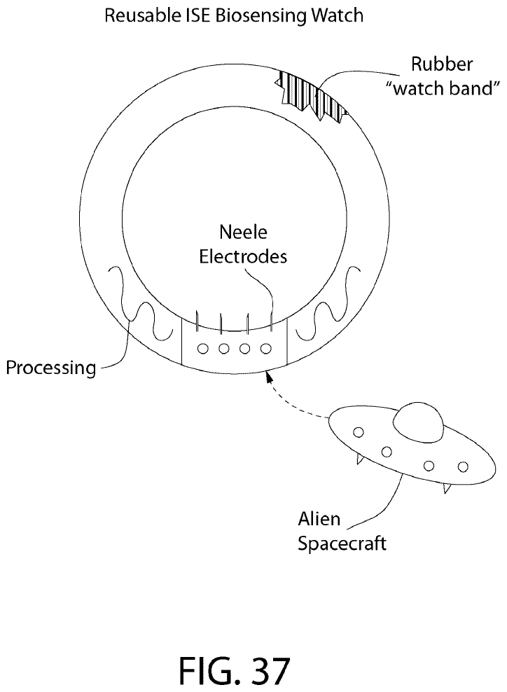

Referring now to FIG. 37, shown is a watch design where sweat is analyzed via needle electrodes with an ISE biosensor. The watch contains circuitry and a BLE connector to an app. The watch is reusable, and the needle component must be sanitized after each use.

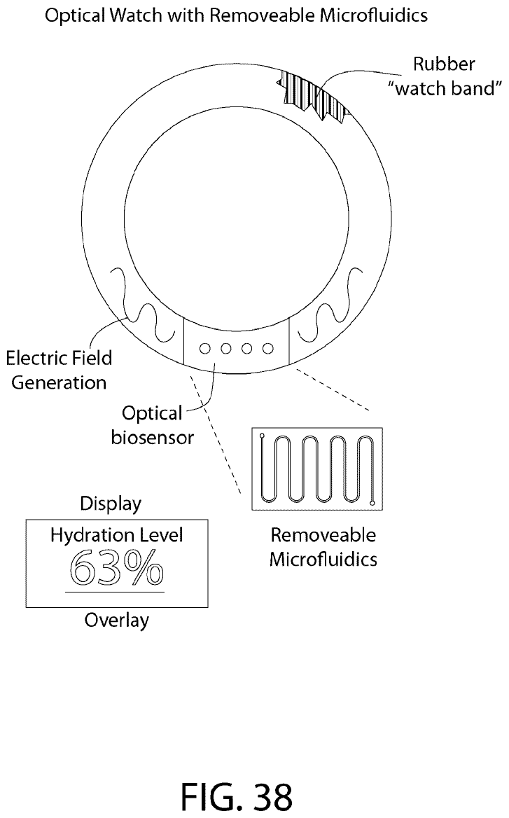

Referring now to FIG. 38, shown is a watch design where sweat is collected via microfluidics and is scanned with an optical biosensor. The watch contains circuitry and provides an electric field to promote sweat generation. The watch is reusable and has a removable microfluidics component. The device includes a display that directly describes hydration levels instead of a BLE connection to an app.

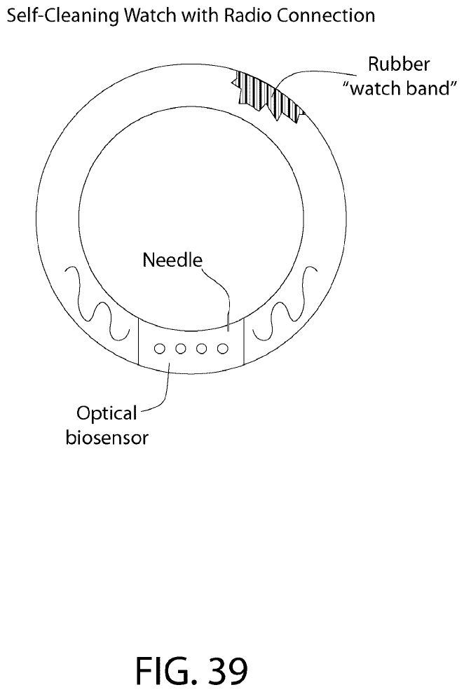

Referring now to FIG. 39, shown is a watch design where sweat is collected via self-cleaning microfluidics and is scanned by an optical biosensor. A radio connection to an alien spacecraft relays when hydration levels are low and the spacecraft beams saline solution through a self-cleaning needle which regulates hydration levels. This idea was constructed as the one “out-there, alien idea” generated during the ideation session and was included to demonstrate creativity.

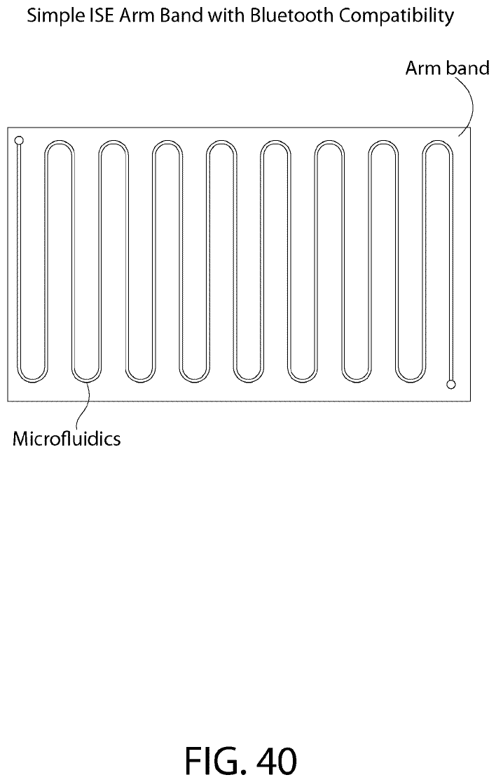

Referring now to FIG. 40, shown is a design that is a basic microfluidic sweat collection ISE biosensor BLE connection to app device that is packaged in the style of a comfortable arm band.

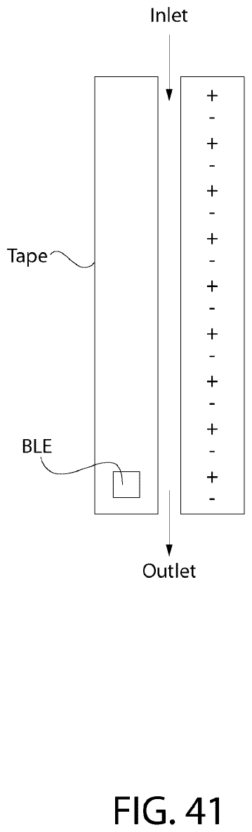

Referring now to FIG. 41, shown is a design that takes the form of flexible adhesive tape that moves sweat through a straight narrow inlet to an outlet. The tape includes a positive and negatively charged field to move sweat through as an ISE biosensor which determines sodium concentration. A BLE adhered to the tape is powered by the movement of sweat through the field and transmits information to an app.

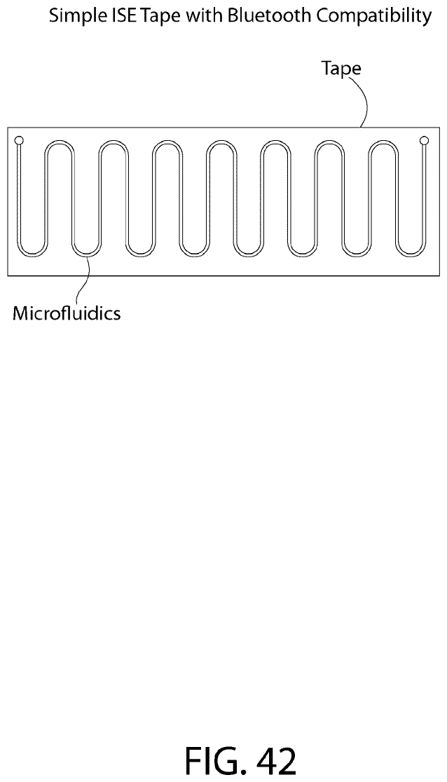

Referring now to FIG. 42, shown is a design that uses microfluidics moving through tape with a basic ISE biosensor and BLE connection to an app to describe hydration levels.

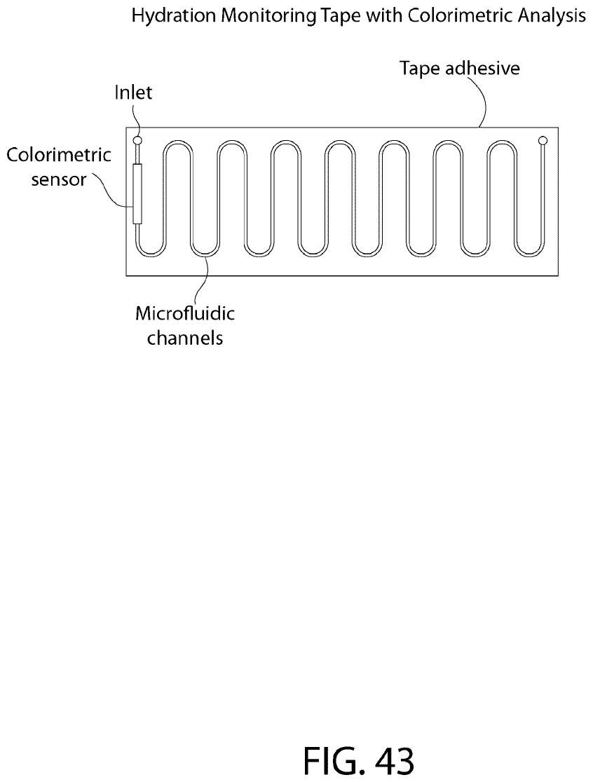

Referring now to FIG. 43, shown is a design that utilizes a tape adhesive, less microchannel loops, and collects sweat in a similar mechanism as previous designs, however it incorporates a colorimetric sensor that can be scanned by an app to reveal hydration levels.

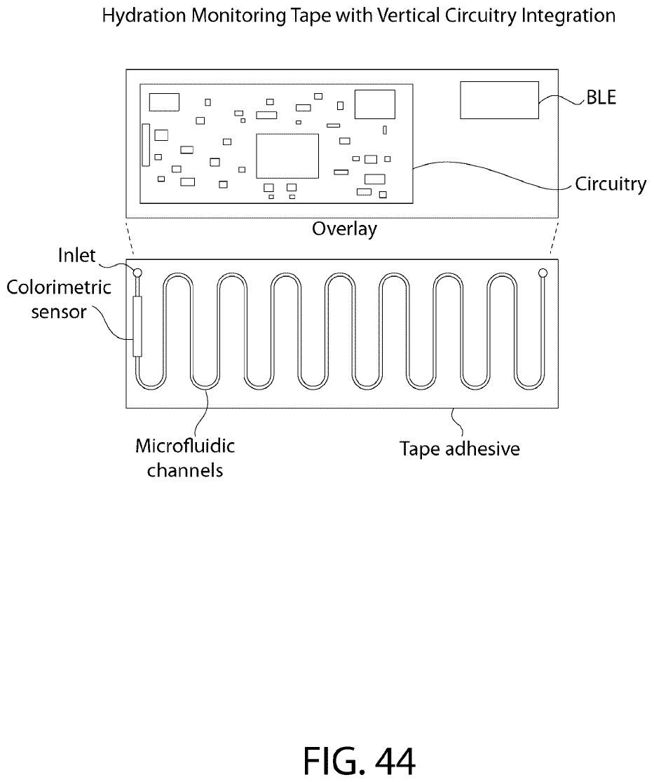

Referring now to FIG. 44, shown is a design that utilizes a tape adhesive, less microchannel loops, and collects sweat in a similar mechanism as previous designs. In contrast to the previous design, it incorporates an overlay to read and process a colorimetric sensor. It contains circuitry and a BLE to connect to an app.

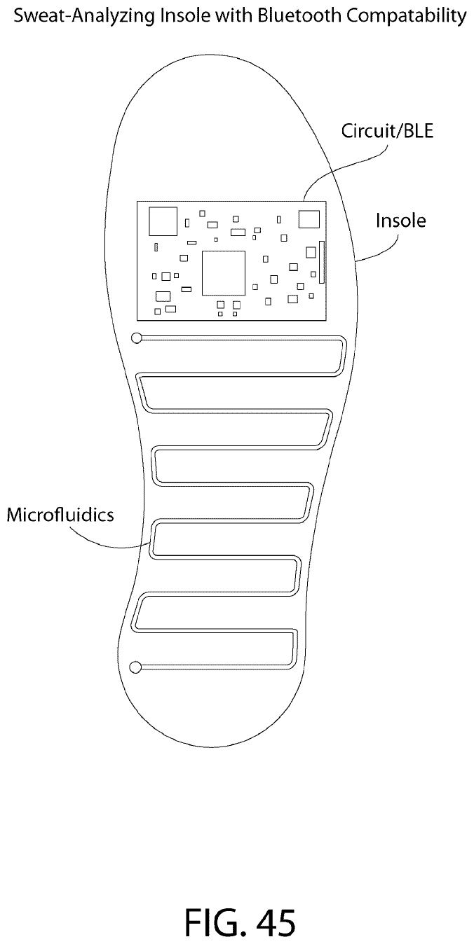

Referring now to FIG. 45, shown is a design that takes advantage of the amount of sweat released by the foot. The design takes the form of a “smart insole” that collects and processes sweat. The insole connects to an app via BLE to relay hydration status.

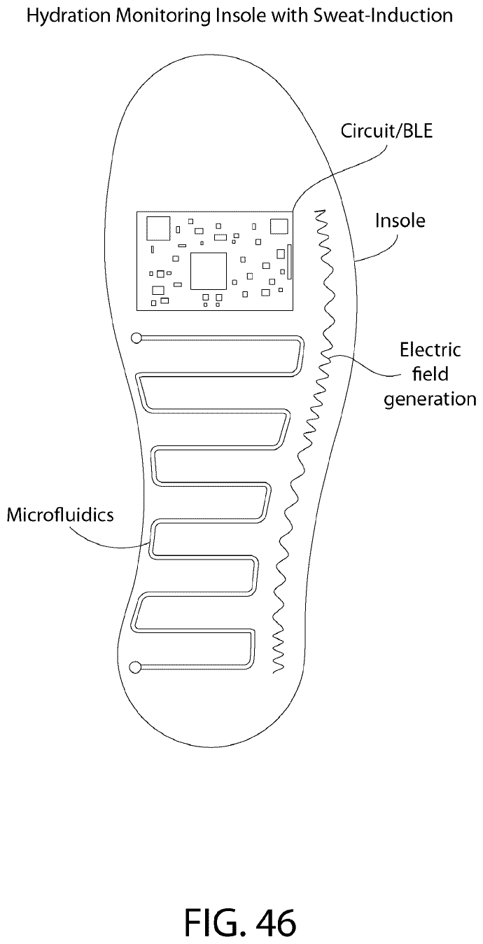

Referring now to FIG. 46, shown is a design that builds off of the last design to collect sweat via a “smart insole” and relay it to an app via BLE. This device includes the additional capabilities of electric field generation to induce sweat on the foot for analysis.

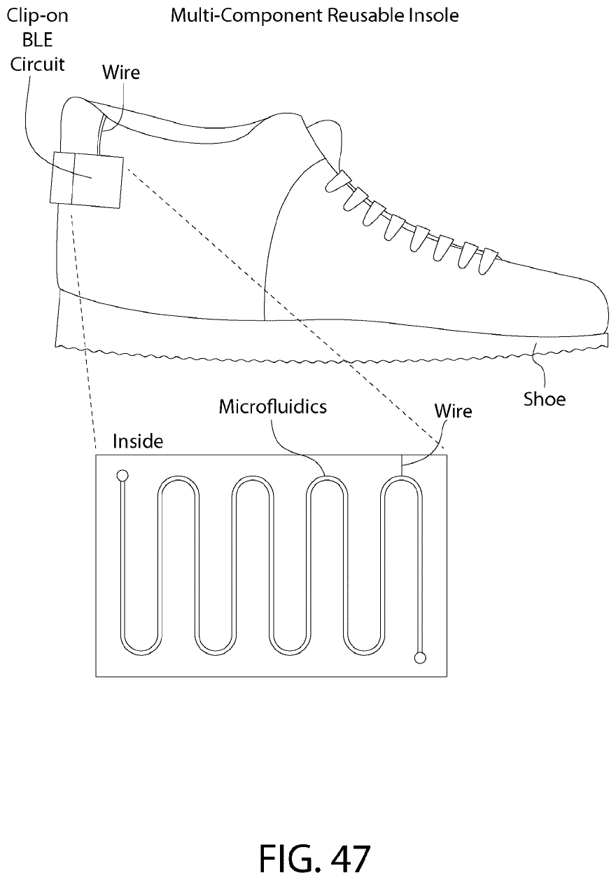

Referring now to FIG. 47, shown is a design that combines the “smart insoles” from ideas 32 and 33 with a reusability component. The insole part will contain disposable microfluidics and a wire will connect it to a clip-on BLE circuit. The sensor will transmit data to an app which will give information about hydration levels.

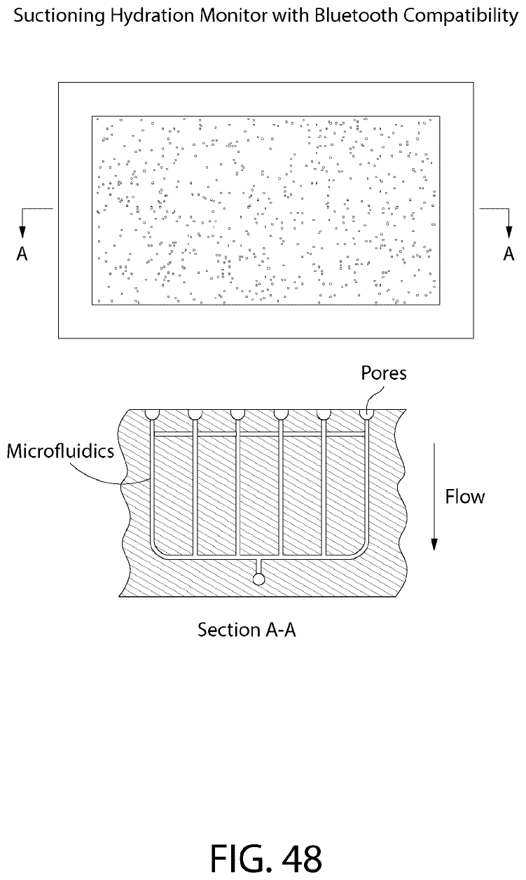

Referring now to FIG. 48, shown is a design that is an adhesive patch with a suctioned component that has multiple inlet “pores” for the microfluidic collection of sweat. The sweat flows further into the patch where a biosensor detects sodium concentration. This data is processed and exported using a BLE circuit to an app that gives information about hydration levels.

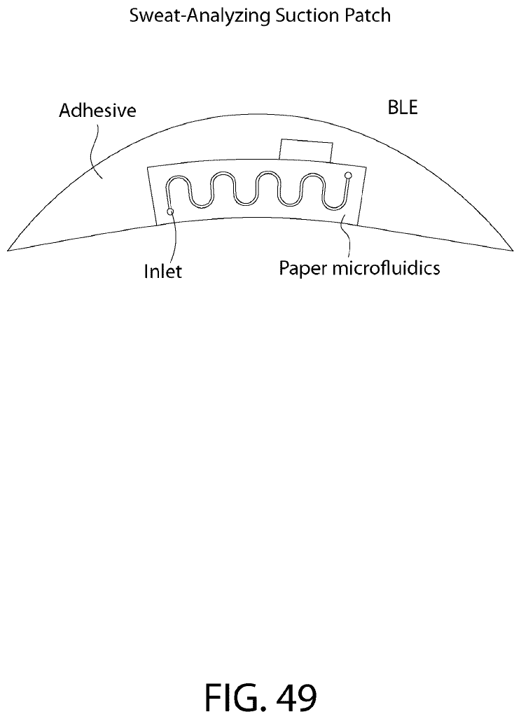

Referring now to FIG. 49, shown is a design that is an adhesive patch curved for suction that uses one inlet and paper microfluidics to collect sweat. A biosensor collects information on sodium concentration in the sweat and a BLE circuit transmits the data to an app.

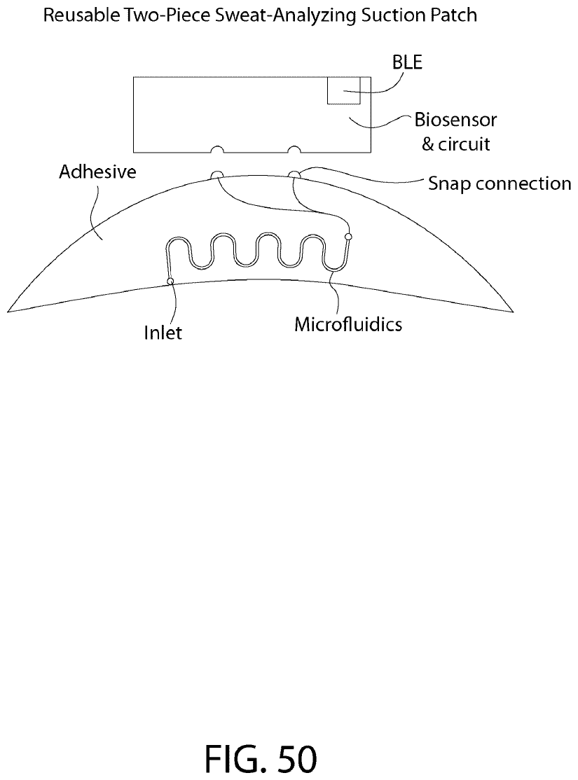

Referring now to FIG. 50, shown is a design that builds off the curved adhesive patch designed previously and adds a reusable biosensor and BLE circuit that attaches by being snapped to the top of the disposable patch.

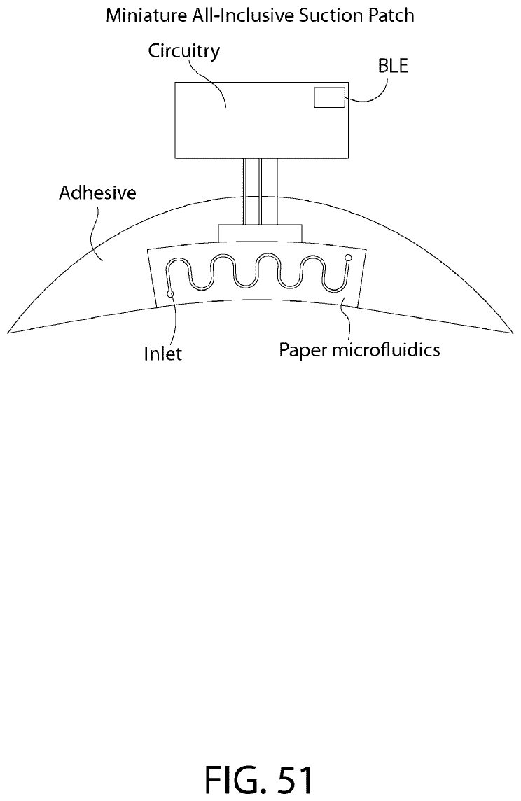

Referring now to FIG. 51, shown is a design that further builds onto the curved adhesive patch by changing the connection type to the reusable circuit and reduces the size of the circuitry to be closer to screen-printed specifications.

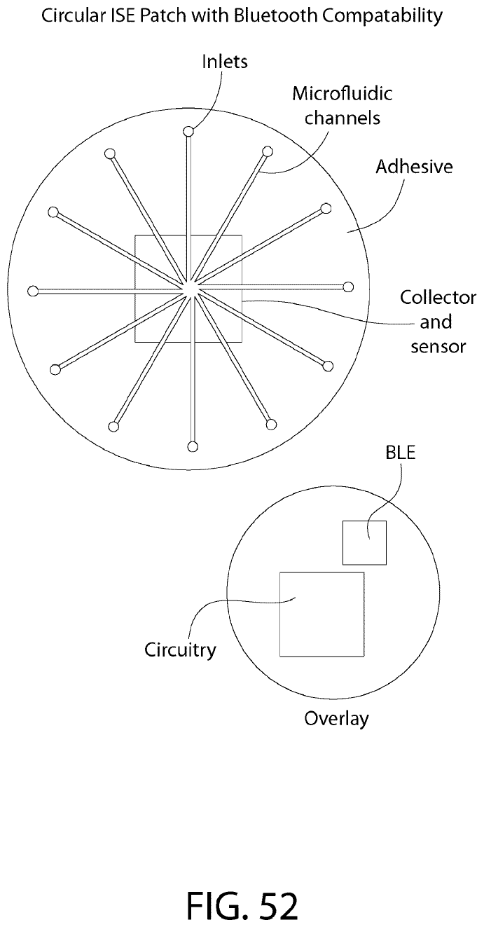

Referring now to FIG. 52, shown is a design that incorporates multiple inlets into a round microfluidic adhesive patch. The sweat is drawn towards the collector and ISE biosensor located at the center of the patch with circuitry and a BLE connection on top. The BLE connects the patch to an app. The entire patch is disposable.

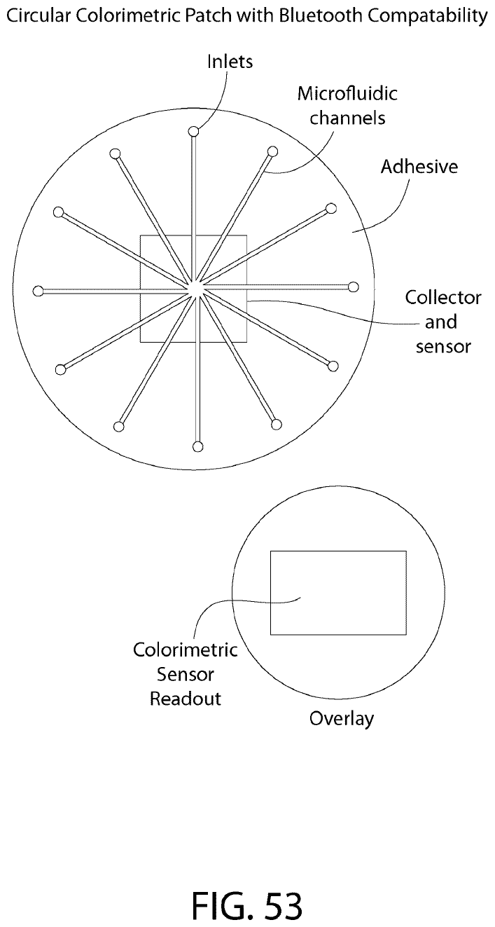

Referring now to FIG. 53, shown is a design that builds off the previous design but makes the adhesive patch cheaper by utilizing a colorimetric sensor above the collection region. This readout can be scanned by a mobile device app to give hydration information for the individual.

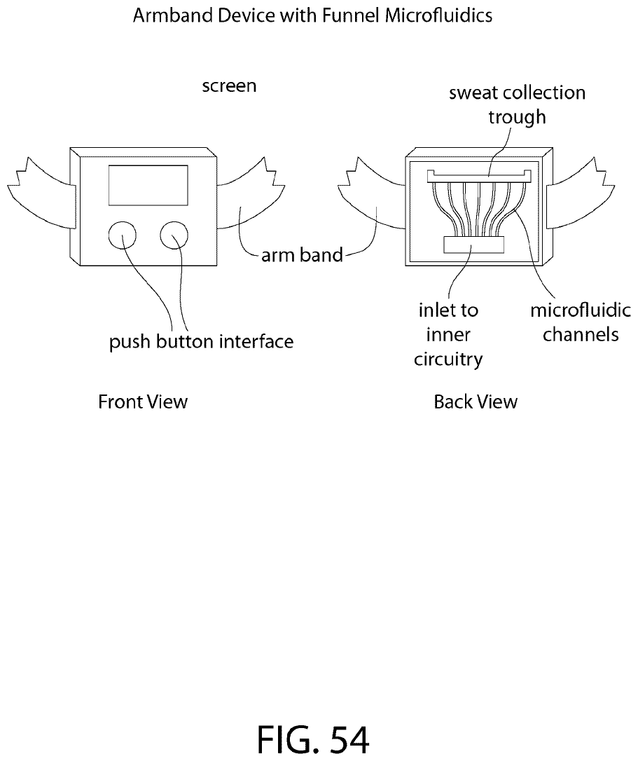

Referring now to FIG. 54, shown is a design that utilizes microfluidics on the back surface of the housing to optimize sweat collection by funneling collected sweat into a reservoir. The microfluidics portion of the design is an adhesive patch, so the device is intended for multiple uses by replacing the microfluidics at each instance. The device is intended to be strapped to the lower bicep region with an elastic band. The small screen on the front of the housing displays readouts from internal sensing circuitry.

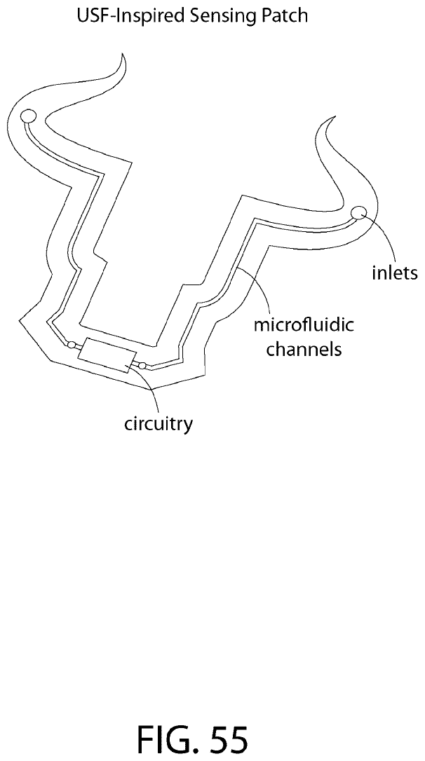

Referring now to FIG. 55, shown is a design that takes the form of a simple patch worn on the surface of the skin, collecting sweat at the microfluidic channel inlets in the tips of the horns. The bilateral nature of this design allows for differential readings to be taken to compare the concentrations sensed in either horn and yield an averaged, ideally more stable signal. Information is intended to be transmitted via Bluetooth to a phone.

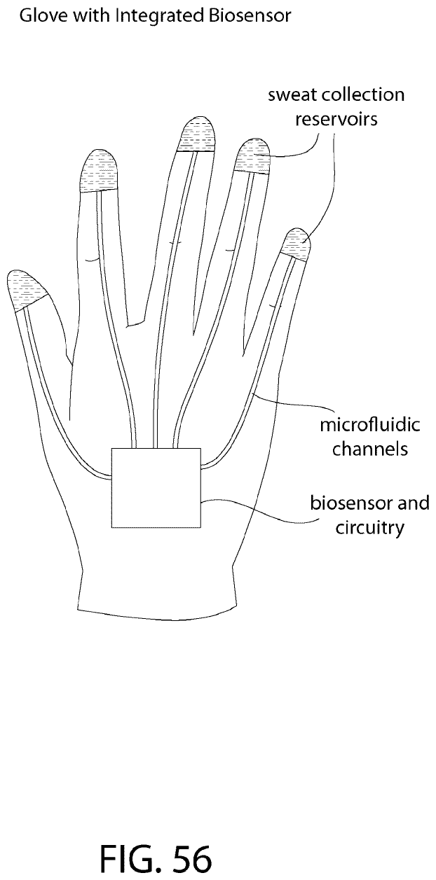

Referring now to FIG. 56, shown is a design that is modeled after a glove, which is intended to maximize sweat collection by trapping heat and collecting all sweat produced by the hands in the fingertip reservoirs. Microfluidic channels running from these reservoirs to the center of the back of the hand transport sweat to a biosensor built on a flexible hybrid electronics patch embedded in the palm region. This design could prove useful in winter applications, where body sweat may be unattainable in cold temperatures, and it also guards against debris and other contaminants by sheltering the sensing region from outside exposure.

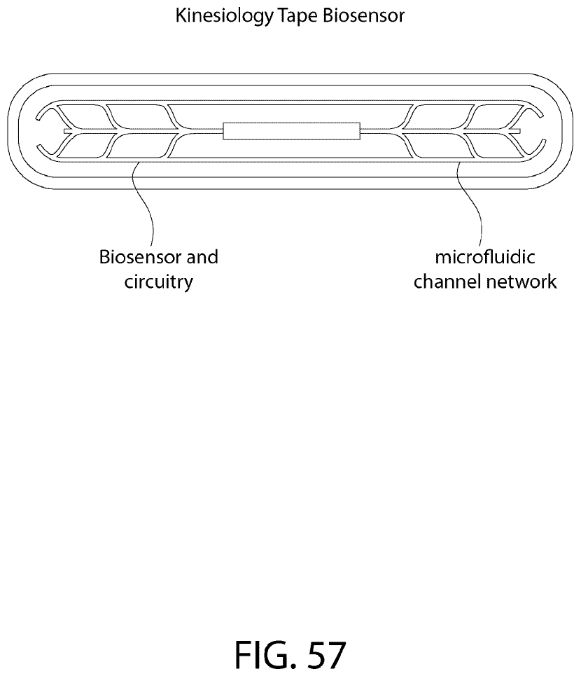

Referring now to FIG. 57, shown is a design that takes advantage of the flexibility and easy application qualities of K-tape incorporated into this sensing tape device. The device can be placed anywhere on the body to personalize the sensing process depending on individuals' most sweaty area. It also functions as a helpful tape for athletes, providing dual functionality. Microfluidic channels run length-wise for maximal collection.

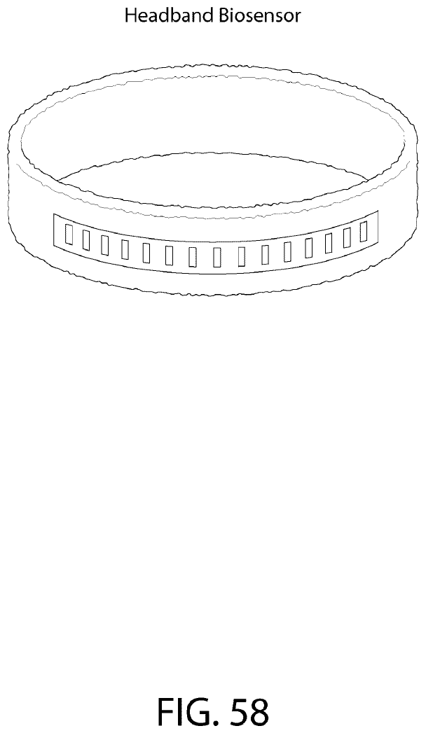

Referring now to FIG. 58, shown is a design that is integrated into a cloth headband, and is intended to be worn on the forehead. It provides colorimetric sensing via a hydration bar on the front, or data can be transmitted to a phone via Bluetooth. The cloth material collects as much sweat as possible via moisture wicking, and it is machine washable for more than one use.

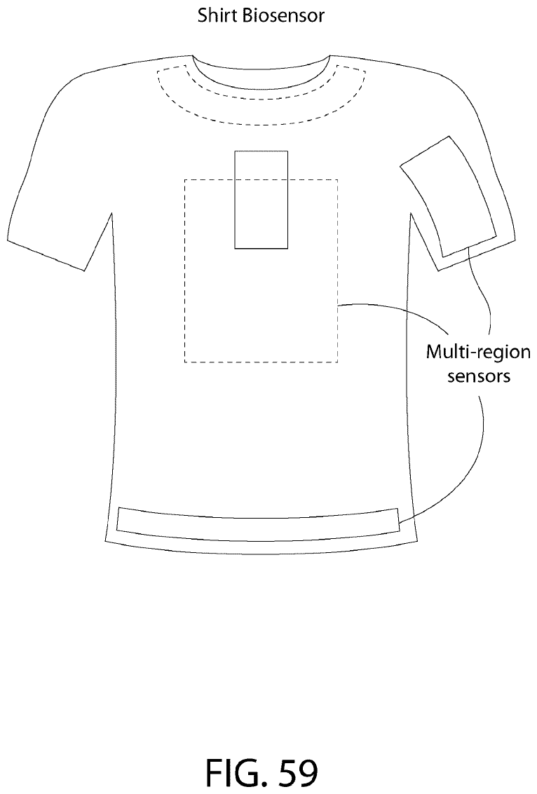

Referring now to FIG. 59, shown is a design that operates via multiple patch-based sensors printed onto fabric using screen-printing techniques. The accompanying circuitry that is housed in the middle of the chest averages regional measurements from each location for more accurate whole-body hydration level characterization. The design could also include optical activation, in which the sweat induces color changes in the fabric.

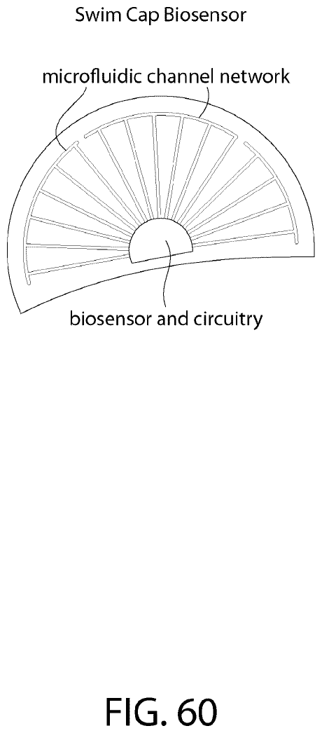

Referring now to FIG. 60, shown is design that is designed specifically for water sport athletes, as the biosensors and circuitry is printed onto polymer substrates to line the inside of the swim cap. This device collects sweat from the hair and is protected from outside contamination.

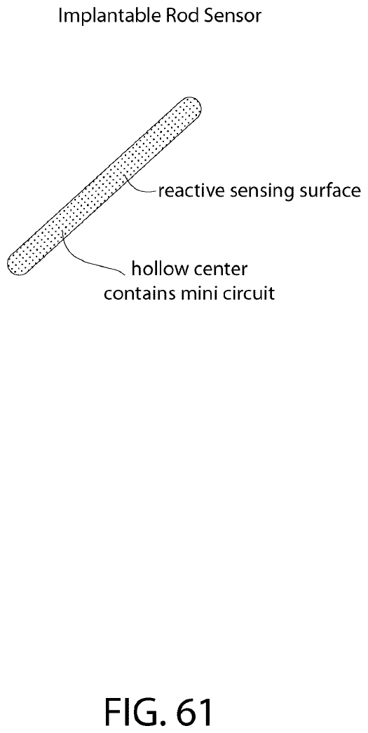

Referring now to FIG. 61, shown is design that is inspired by common birth control methods, employing subcutaneous sensing which provides more accurate correlations as eccrine sweat is diluted by the skin. It is intended as a long-term solution and meant to be used for extended periods of time. Miniature circuitry built into the hollow center of the rod allows for NFC reading of data via a phone.

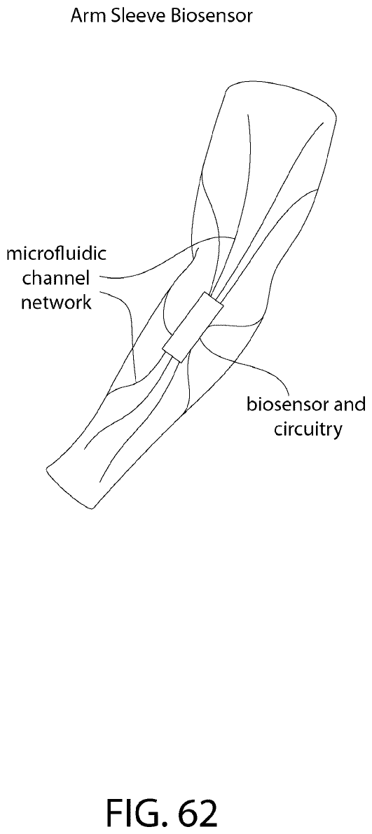

Referring now to FIG. 62, shown is a design that is inspired by basketball athletes, who tend to sweat quite a lot during the course of a game. Microfluidic channels crisscross from the external portion of the forearm to wrap around the upper arm and along the length of the forearm. The channels converge to a chip embedded in the sleeve. This device is machine washable and intended for multiple uses.

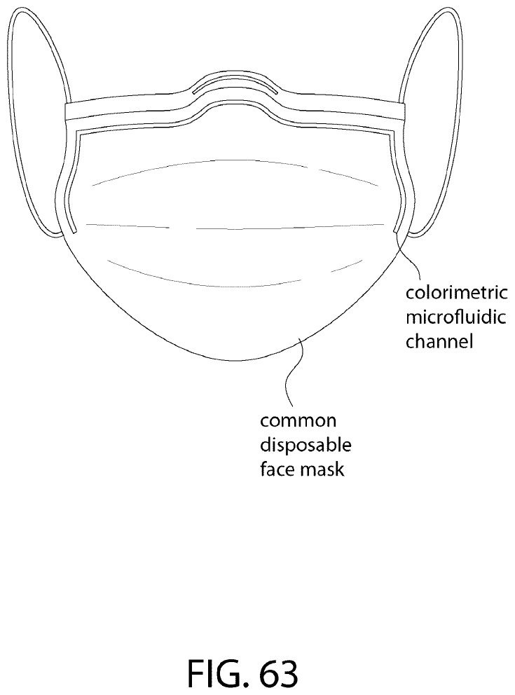

Referring now to FIG. 63, shown is a design that functions with sensing regions contacting the face along the boundaries of the mask that tend to sit close to the skin. The absorbent material around the edges of the mask collects sweat to transport it to a biosensor integrated into the fabric, and data is accessed via Bluetooth transmission.

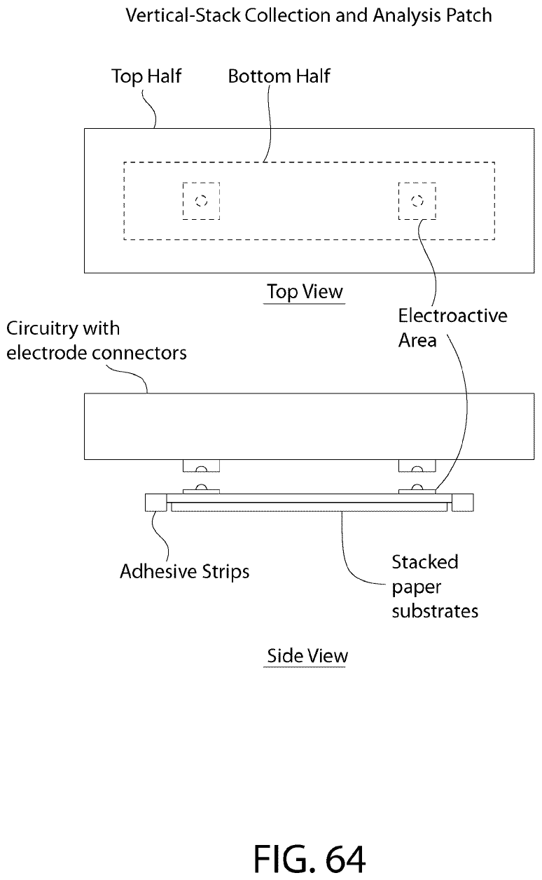

Referring now to FIG. 64, shown is a design that is based on stacked paper substrates, which act as sponges to wick sweat from the skin's surface and deliver it vertically to the sensing areas of the device. Hydrophobic patterns on the stacked paper substrate layers allows for 3D microfluidics by controlling vertical flow and limiting lateral spread. The electroactive area is defined by the connectors' borders to directly connect to circuitry. The design comprises a reusable top half with a cheap disposable collecting element on the bottom half.

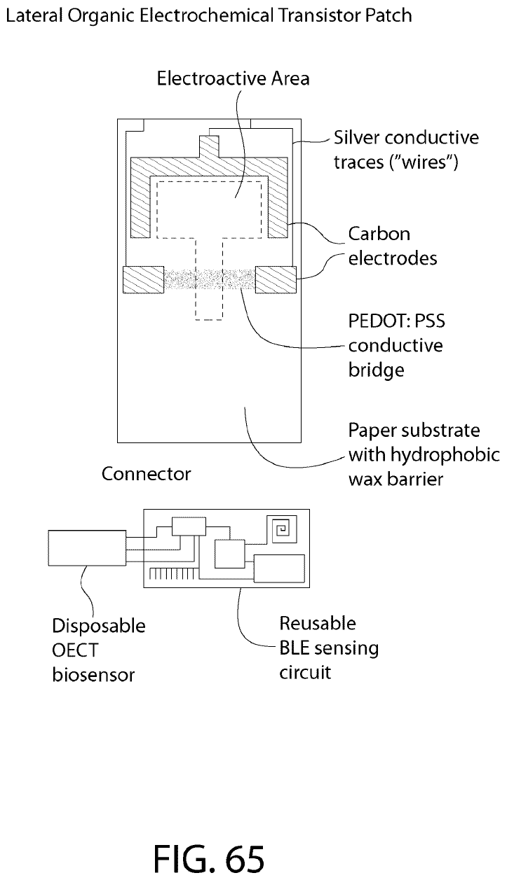

Referring now to FIG. 65, shown is a design that is based on paper-based microfluidics, with a microfluidic channel defined by hydrophobic areas created by wax printing. The paper substrate functions to collect and sense the desired analyte in one unit, but this element is cheap and disposable and connects to multi-use circuitry via Crimpflex connectors.

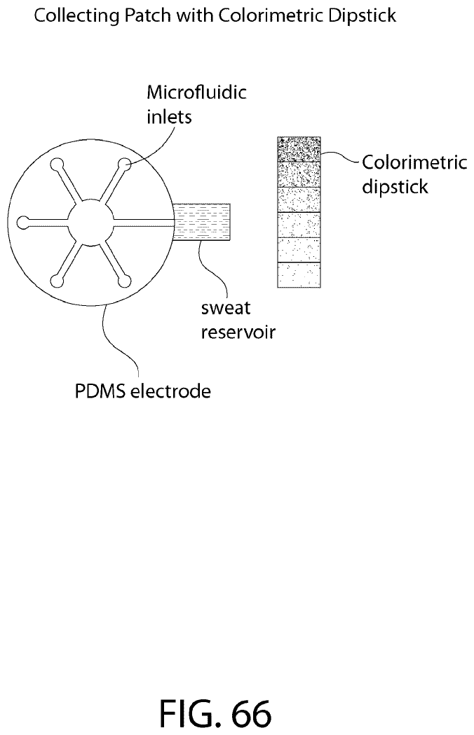

Referring now to FIG. 66, shown is a design that operates via PDMS-casted microfluidic channels to capture sweat in a central reservoir, which is then squeezed to release the sweat contents onto chemically-treated chromatography paper strips to yield a color-based concentration analysis. The distance of the furthest activated region on the strip indicates the user's hydration level.

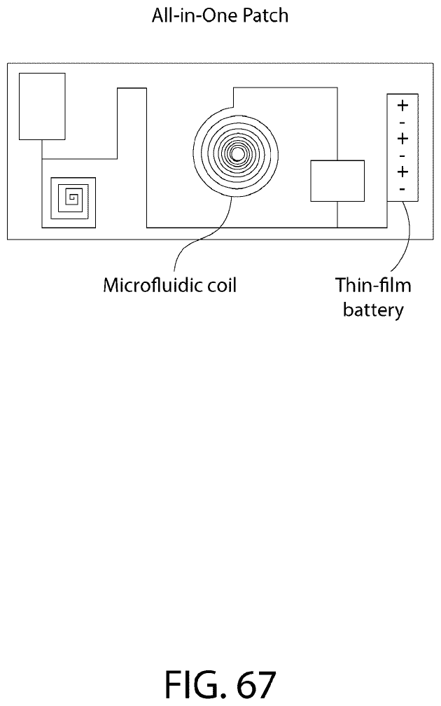

Referring now to FIG. 67, shown is a design that contains microfluidic channels integrated into a screen-printed patch on a flexible substrate, which enables placement at various locations on the body. The circuitry accompanying this design conducts minimal signal processing, opting instead to transmit data to a device via Bluetooth. A thin-film battery that is also screen-printed enables one-time usage of this patch at a low cost.

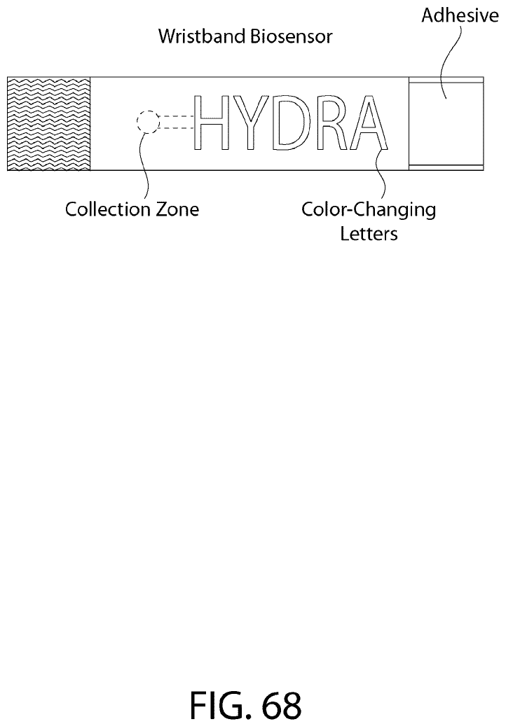

Referring now to FIG. 68, shown is a design that employs paper microfluidics with the substrate sandwiched between encapsulant layers that define hydrophilic areas. Wax hydrophobic barriers speed up fluid delivery into the lettering for colorimetric concentration analysis. The operation of this design yields quick and simple detection, albeit with less precise or meaningful readouts.

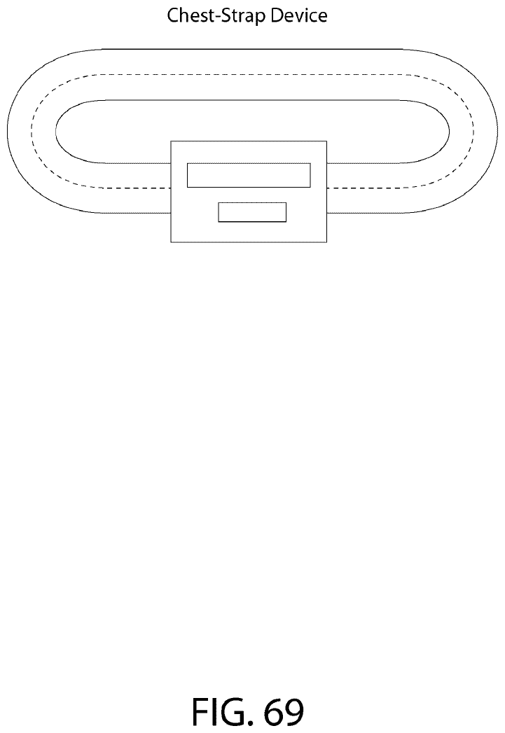

Referring now to FIG. 69, shown is a design that is intended to be worn around the chest, with the strap containing microfluidic channels. All of the sweat dripping down from the front and back surface of the individual is collected by the strap channels and transported to the device located centrally in the housing. This design is ultimately inspired by athletes, who wear sports performance trackers quite frequently, such as soccer players.

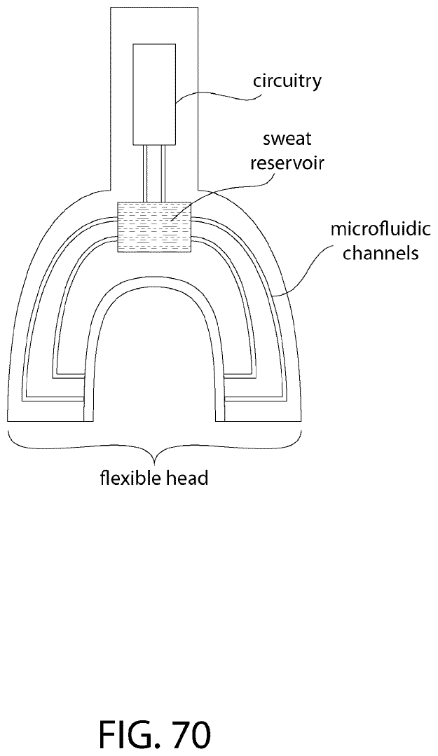

Referring now to FIG. 70, shown is a design that is based on a squeegee with a flexible membrane in the concave region. The intended usage involves wiping the arms and legs with the concave region to collect all surface sweat into a reservoir. Circuitry is located in the handle to analyze concentration, and the device can be flushed for reuse.

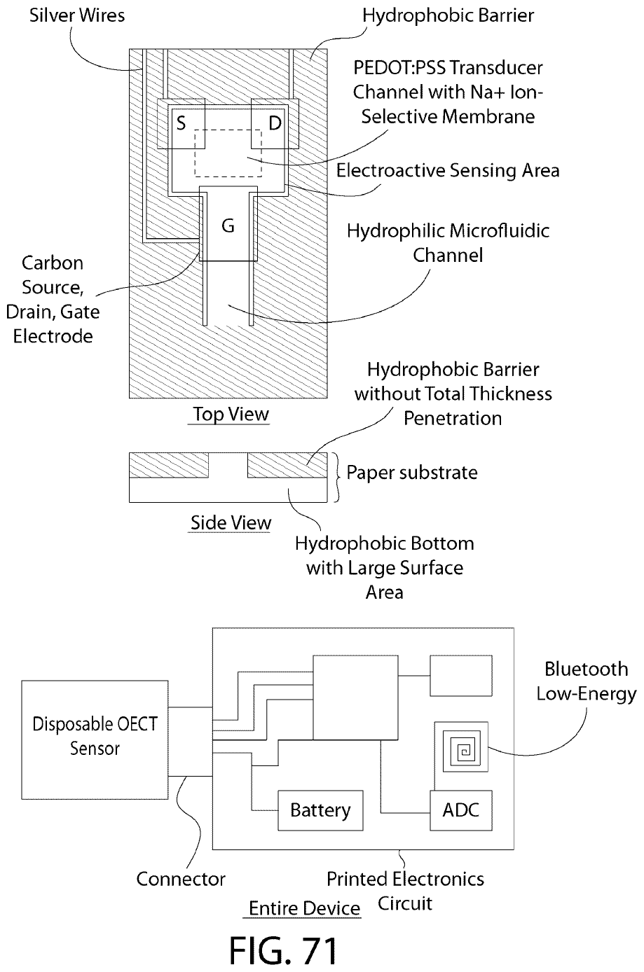

Referring now to FIG. 71, shown is a design that is based on the organic electrochemical transistor biosensing technology that will form the basis of our proposed solution, and it is one of the three proposed solutions briefed in this report.

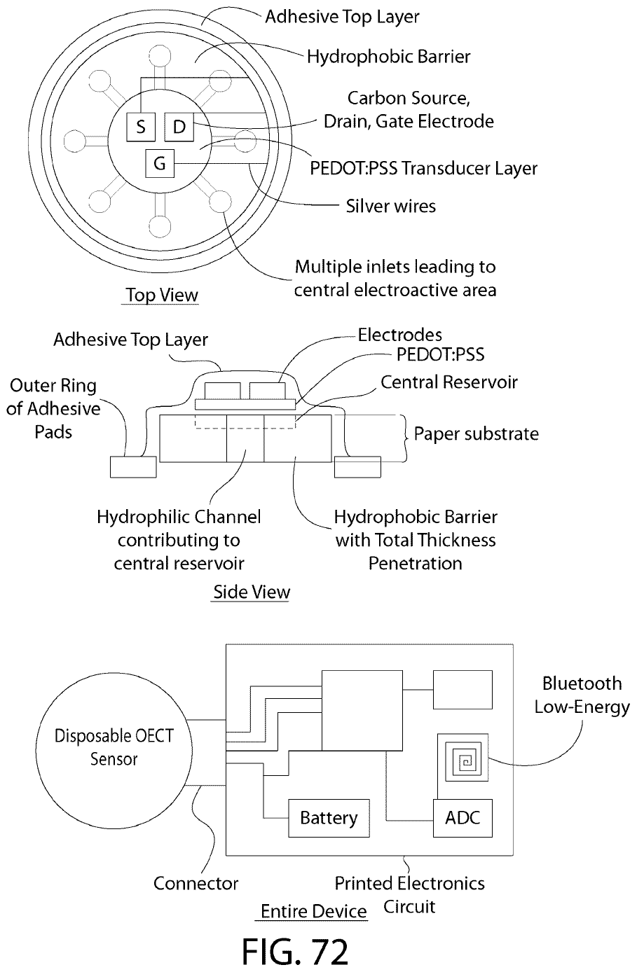

Referring now to FIG. 72, shown is a design that is unique to the other two proposed solutions detailed in this report, as it takes a circular shape and employs multiple inlets for a central reservoir. It nevertheless employs the same organic electrochemical transistor biosensing technology.

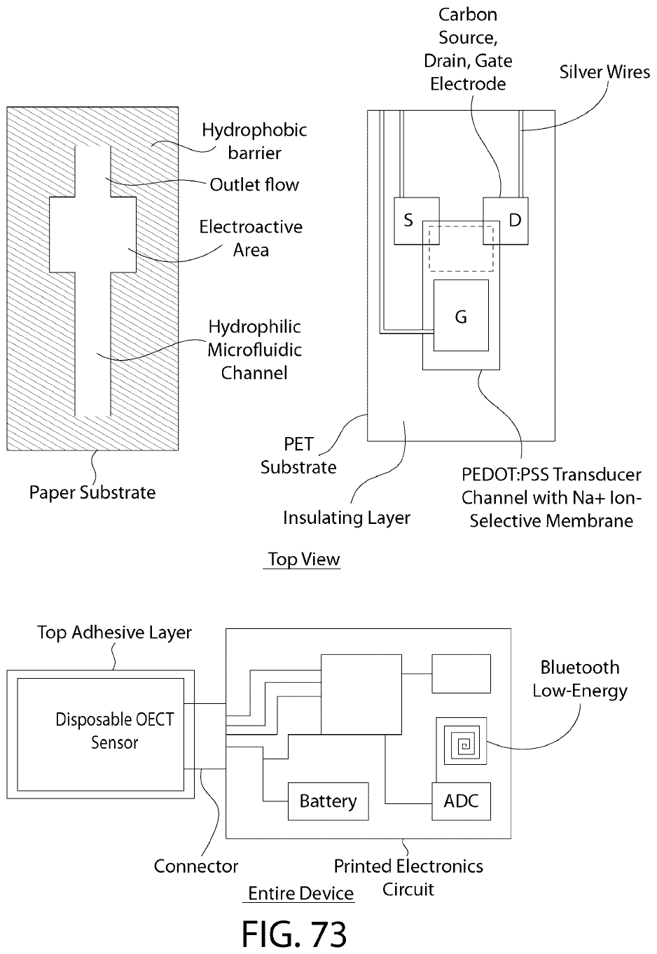

Referring now to FIG. 73, shown is a design that was one of the three proposed solutions presented in this report, and ultimately inspired the final selected design.

In the foregoing Detailed Description, it can be seen that various features are grouped together in a single embodiment for the purpose of clarity and brevity of the disclosure. This method of disclosure is not to be interpreted as reflecting an intention that the embodiments require more features than are expressly recited herein. Rather, the disclosure is to encompass all variations and modifications to the disclosed embodiments that would be understood to the skilled artisan in light of the disclosure.

What is claimed is:

1. A hydration detection device, comprising:
a flexible substrate having screen-printed therein an electrical circuit comprising a plurality of layers of conductive and dielectric inks;
a sensor upon the flexible substrate, wherein the sensor is capable of detecting element data indicative of characteristics of sweat;
untreated chromatographic paper placed so as to contact skin of a user, and capable of wicking the sweat from the skin to the sensor along a micro-sized fluidic channel; and
a bluetooth antenna on the flexible substrate and communicative with the sensor, and capable of communicating the element data wirelessly to an application capable of indicating to the user a propriety of the hydration of the user based upon the element data.
